(19) United States
(12) Patent Application Publication
(10) Pub. No.: US 2023/0172997 A1
(43) Pub. Date: Jun. 8, 2023

(54) RECOMBINANT BACTERIA FOR PRODUCTION OF INDOLE-3-ACETIC ACID (IAA) AND USES THEREOF

(71) Applicant: Synlogic Operating Company, Inc., Cambridge, MA (US)

(72) Inventors: Ning Li, Winchester, MA (US); Jian-Rong Gao, Malden, MA (US); Christopher George Bergeron, Fitchburg, MA (US)

(21) Appl. No.: 17/923,955

(22) PCT Filed: May 26, 2021
(86) PCT No.: PCT/US2021/034314

§ 371 Date: Nov. 8, 2022

Related U.S. Application Data
(60) Provisional application No. 63/030,135, filed on May 26, 2020.

(51) Int. Cl. A61K 35/74; C12N 1/20; C12N 15/52; C12N 15/70; C12P 17/10; C12N 9/06; C12N 9/10; C12N 9/88; A61P 3/00

(52) U.S. Cl. CPC A61K 35/74; C12N 1/20; C12N 15/52; C12N 15/70; C12P 17/10; C12N 9/0016; C12N 9/1077; C12N 9/1085; C12N 9/88; A61P 3/00; C12Y 402/0102; C12Y 401/01074; C12Y 104/01019; C12Y 205/01054; C12Y 401/03027; C12Y 401/01048; C12Y 204/02018; C12R 2001/19

(57) ABSTRACT

The present disclosure provides recombinant bacteria for production of indole-3-acetic acid (IAA). Pharmaceutical compositions and methods of treating diseases are also included.

### RELATED APPLICATIONS

The instant application claims priority to U.S. Provisional Application No. 63/030,135 filed May 26, 2020, entire contents of which are expressly incorporated by reference herein in its entirety.

### SEQUENCE LISTING

The instant application contains a Sequence Listing which has been submitted electronically in ASCII format and is hereby incorporated by reference in its entirety. Said ASCII copy, created on May 11, 2021, is named 126046-05720_SL.txt and is 53,325 bytes in size.

### BACKGROUND

Nonalcoholic fatty liver disease (NAFLD), the most common chronic liver disease worldwide, has been known to be inherently associated with other metabolic diseases such as obesity, insulin resistance, and dyslipidemia (Norheim F, et al., J Lipid Res. 2017; 58(1):178-187). NAFLD is defined as the presence of hepatic steatosis on liver biopsy that is not initiated by alcohol consumption or other reasons (e.g., drugs, toxins, infections) (Abd El-Kader S M, E et al., World JHepatol. 2015; 7(6):846-58). Currently, the prevalence rate of NAFLD is estimated to be 24% around the world, among which 5-20% of patients with simple steatosis progress to nonalcoholic steatohepatitis (NASH). NASH is characterized by steatosis with lobular inflammation and the ballooning of hepatocytes, and increased risk of fibrosis, cirrhosis, and hepatocellular carcinoma (Takahashi Y, Fukusato T, World J Gastroenterol. 2014; 20(42): 15539-48).

A large amount of evidence has revealed that the gut microbiota plays vital roles in regulation of NAFLD via producing bacterial metabolites (Roager H M, Licht T R, Nat Commun. 2018; 9(1):3294; Ji Y, et al., Nutrients. 2019; 11(8)). Dietary tryptophan can be metabolized into indole-3-acetic acid (IAA) by gut microbiota through indole-3-acetamide pathway under the catalysis of tryptophan monooxygenase and indole-3-acetamide hydrolase (Hubbard T D, et al., Drug Metab Dispos. 2015; 43(10):1522-35). Previous in vitro study demonstrated that IAA possessed the ability of scavenging free radicals (Kim D, et al., Oxid Med Cell Longev. 2017: 8639485). Additionally, results from cultured cell lines of macrophages and hepatocytes indicated that IAA mitigates pro-inflammatory cytokine production from macrophages exposed to endotoxin and attenuates lipogenesis in hepatocytes induced by cytokine and free fatty acids (Krishnan S, et al., Cell Rep. 2018; 23(4):1099-1111). Recently, the protective effects of IAA on high-fat diet-induced NAFLD have been reported in an in vivo study, demonstrating that IAA alleviates high-fat diet-induced hepatotoxicity in mice, which proves to be associated with the amelioration in insulin resistance, lipid metabolism, and oxidative and inflammatory stress (Ji Y., et al., Nutrients. 2019; 11(9): 2062).

Currently, there is no accepted approach to treating NAFLD and NASH. Therapy generally involves treating known risk factors such as correction of obesity through diet and exercise, treating hyperglycemia through diet and insulin, avoiding alcohol consumption, and avoiding unnecessary medication. Given the critical role that IAA plays in the development of liver diseases, IAA constitutes an important therapeutic target.

Accordingly, there exists an ongoing need for novel compositions for producing IAA in order to treat and/or prevent liver diseases and other associated metabolic diseases.

### SUMMARY

The present disclosure provides a recombinant bacteria for production of indole-3-acetic acid (IAA), pharmaceutical compositions thereof, and methods of modulating and treating diseases. The recombinant bacteria are capable of producing indole-3-acetic acid in low-oxygen environments, e.g., the gut. Thus, the recombinant bacteria and pharmaceutical compositions comprising those bacteria are non-pathogenic, and can be used in order to treat and/or prevent conditions associated with diseases, including obesity, diabetes, and liver diseases.

The disclosure provides, in one aspect, a recombinant bacterium comprising one or more gene cassettes for producing indole 3-acetic acid (IAA), wherein the one or more gene cassette comprises one or more sequences encoding the genes selected from the group consisting of trpE^{fbr}, trpB, trpC, trpD and trpA, and wherein the one or more gene cassettes are operably linked to a directly or indirectly inducible promoter that is not associated with the gene cassettes and is induced by exogenous environmental conditions. In one embodiment, the one or more sequences are heterologous sequences.

In some embodiments, the gene sequence encoding TrpE has at least 90%, 91%, 92%, 93%, 94%, 95%, 96%, 97%, 98%, 99% identity to, comprises, or consists of SEQ ID NO: 1, wherein the gene sequence encoding TrpB has at least 90%, 91%, 92%, 93%, 94%, 95%, 96%, 97%, 98%, 99% identity to, comprises, or consists of SEQ ID NO: 2, wherein the gene sequence encoding TrpC has at least 90%, 91%, 92%, 93%, 94%, 95%, 96%, 97%, 98%, 99% identity to, comprises, or consists of SEQ ID NO: 3, wherein the gene sequence encoding TrpD has at least 90%, 91%, 92%, 93%, 94%, 95%, 96%, 97%, 98%, 99% identity to, comprises, or consists of SEQ ID NO: 4, and/or wherein the gene sequence encoding TrpA has at least 90%, 91%, 92%, 93%, 94%, 95%, 96%, 97%, 98%, 99% identity to, comprises, or consists of SEQ ID NO: 5.

In some embodiments, the amino acid sequence of TrpE has at least 90%, 91%, 92%, 93%, 94%, 95%, 96%, 97%, 98%, 99% identity to, comprises, or consists of SEQ ID NO: 14, wherein the amino acid sequence of TrpB has at least 90%, 91%, 92%, 93%, 94%, 95%, 96%, 97%, 98%, 99% identity to, comprises, or consists of SEQ ID NO: 15, wherein the amino acid sequence of TrpC has at least 90%, 91%, 92%, 93%, 94%, 95%, 96%, 97%, 98%, 99% identity to, comprises, or consists of SEQ ID NO: 16, wherein amino acid sequence of TrpD has at least 90%, 91%, 92%, 93%, 94%, 95%, 96%, 97%, 98%, 99% identity to, comprises, or consists of SEQ ID NO: 17, and/or wherein amino acid sequence of TrpA has at least 90%, 91%, 92%, 93%, 94%, 95%, 96%, 97%, 98%, 99% identity to, comprises, or consists of SEQ ID NO: 18.

In some embodiments, the recombinant bacterium further comprises a second gene cassette, wherein the second gene cassette comprises one or more sequences encoding the genes selected from the group consisting of aroG^{fbr}, trpDH, ipdC and iad1. In some embodiments, the second gene cassette comprises sequences encoding AroG, TrpDH, IpdC, and Iad1. In one embodiment, the one or more sequences are heterologous sequences.

In some embodiments, the gene sequence encoding AroG has at least 90%, 91%, 92%, 93%, 94%, 95%, 96%, 97%, 98%, 99% identity to, comprises, or consists of SEQ ID NO: 6 wherein the gene sequence encoding TrpDH has at least 90%, 91%, 92%, 93%, 94%, 95%, 96%, 97%, 98%, 99% identity to, comprises, or consists of SEQ ID NO: 7, wherein the gene sequence encoding IpdC has at least 90%, 91%, 92%, 93%, 94%, 95%, 96%, 97%, 98%, 99% identity to, comprises, or consists of SEQ ID NO: 8, and/or wherein the gene sequence encoding Iad1 has at least 90%, 91%, 92%, 93%, 94%, 95%, 96%, 97%, 98%, 99% identity to, comprises, or consists of SEQ ID NO: 9.

In some embodiments, the amino acid sequence of AroG has at least 90%, 91%, 92%, 93%, 94%, 95%, 96%, 97%, 98%, 99% identity to, comprises, or consists of SEQ ID NO: 19, wherein the amino acid sequence of TrpDH has at least 90%, 91%, 92%, 93%, 94%, 95%, 96%, 97%, 98%, 99% identity to, comprises, or consists of SEQ ID NO: 20, wherein the amino acid sequence of IpdC has at least 90%, 91%, 92%, 93%, 94%, 95%, 96%, 97%, 98%, 99% identity to, comprises, or consists of SEQ ID NO: 21, and/or wherein the amino acid sequence of Iad1 has at least 90%, 91%, 92%, 93%, 94%, 95%, 96%, 97%, 98%, 99% identity to, comprises, or consists of SEQ ID NO: 22.

In other embodiments, the recombinant bacterium further comprises a ribosome binding site before the aroG^{fbr} gene, the trpDH gene, the ipdC gene, and/or the iad1 gene.

In some embodiments, the bacterium further comprises a deletion or a mutation in a gene encoding tryptophan transcriptional repressor (TrpR) and/or a deletion or a mutation in a gene encoding tryptophanase (TnaA).

In some embodiments, the promoter is directly or indirectly induced by low-oxygen or anaerobic conditions.

In other embodiments, the promoter is an FNR-inducible promoter.

In some embodiments, the one or more gene cassettes and operatively linked promoter are present on a plasmid in the bacterium. In other embodiments, the one or more gene cassettes and operatively linked promoter are present on a chromosome in the bacterium.

In some embodiments, the recombinant bacterium comprises a deletion or mutation in a gene encoding tryptophan transcriptional repressor (TrpR). In other embodiments, the recombinant bacterium comprises a deletion or mutation in a gene encoding tryptophanase (TnaA).

In some embodiments, the recombinant bacterium is a non-pathogenic bacterium. In other embodiments, the recombinant bacterium is a probiotic or a commensal bacterium.

In some embodiments, the recombinant bacterium is selected from the group consisting of Bacteroides, Bifidobacterium, Clostridium, Escherichia, Lactobacillus, and Lactococcus. In other embodiments, the recombinant bacterium is Escherichia coli strain Nissle.

In some embodiments, the bacterium is capable of producing about 1 μM indole-3-acetic acid (IAA) to about 200 μM IAA. In some embodiments, the bacterium is capable of producing about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 110 μM, about 120 μM, about 130 μM, about 140 μM, about 150 μM, about 160 μM, about 170 μM, about 180 μM, about 190 μM or about 200 μM IAA. In some embodiments, the bacterium is capable of producing about 2-200 μM, about 5-150 μM, about 5-100 μM, about 10-100 μM, about 20-100 μM, about 20-80 μM, about 30-75 μM or about 5-80 μM IAA.

In some embodiments, the bacterium is capable of producing about 1 μM tryptophan to about 200 μM tryptophan. In some embodiments, the bacterium is capable of producing about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 110 μM, about 120 μM, about 130 μM, about 140 μM, about 150 μM, about 160 μM, about 170 μM, about 180 μM, about 190 μM or about 200 μM tryptophan. In some embodiments, the bacterium is capable of producing about 2-200 μM, about 5-150 μM, about 5-100 μM, about 10-100 μM, about 20-100 μM, about 20-80 μM, about 30-75 μM or about 5-80 μM tryptophan.

In another aspect, the invention provides a pharmaceutically acceptable composition comprising the bacterium as described herein; and a pharmaceutically acceptable carrier.

In some embodiments, the pharmaceutically acceptable composition is formulated for oral administration.

In one aspect, the invention provides a method of treating a disease in a subject in need thereof. The method comprises the step of administering to the subject the pharmaceutical composition as described herein.

In one embodiment, the disease or disorder is a liver disease, a metabolic disease, or an autoimmune diseases. In some embodiments, the disorder is selected from the group consisting of: liver disease; non-alcoholic fatty liver disease (NAFLD); non-alcoholic steatohepatitis (NASH); liver cirrhosis; obesity; type 1 diabetes; type 2 diabetes; metabolic syndrome; Bardet-Biedel syndrome; Prader-Willi syndrome; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.

In some embodiments, the subject has an increased level of IAA after the composition is administrated. In other embodiments, the subject has a decreased level of tryptophan after the composition is administrated. In some embodiments, the subject is a human.

### BRIEF DESCRIPTION OF THE FIGURES

FIG. 1A depicts a schematic of an exemplary engineered bacterium for the production of tryptamine. FIG. 1B depicts a schematic of an exemplary engineered bacterium for the production of indole-3-acetic acid (IAA). TrpR is tryptophan transcriptional repressor, and TnaA is tryptophanase or tryptophan indole-lyase.

FIG. 2A depicts production of IAA by engineered bacterial strains at different induction time points (T0=0 h; T2=2 h; and T4=4 h). FIG. 2B depicts production of tryptophan by engineered bacterial strains at different induction time points (T0=0 h; T2=2 h; and T4=4 h). 2342 SFL was the original strain made in SFL. 2342 Run 10 was remade strain by transformed the plasmids described herein into the host strain of 2126, PD manufactured. LLOQ (um): IAA 4.57, Trp 3.92.

FIG. 3 depicts IAA production from PD made samples. LLOQ (um) for IAA was 0.91.

FIG. 4A depicts aryl hydrocarbon receptor (AHR) agonist producing strains. FIG. 4B depicts IAA production from enzyme activity balanced strains.

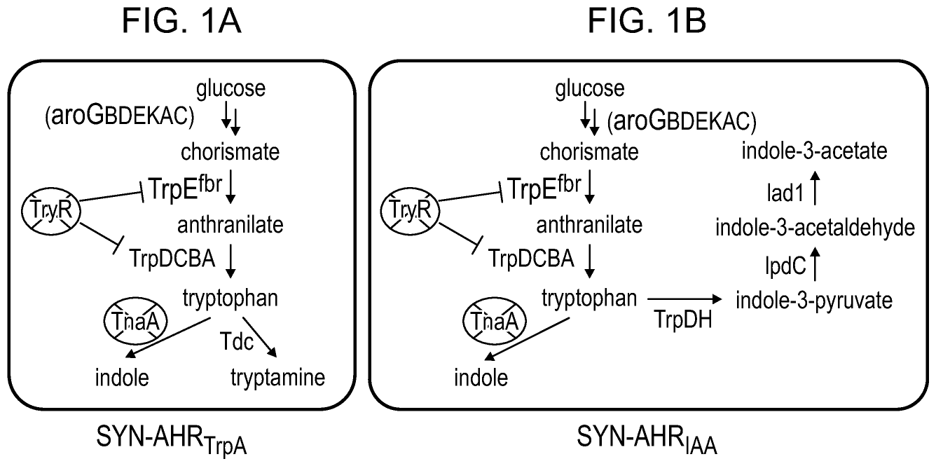

### DETAILED DESCRIPTION

The present disclosure provides recombinant bacteria for production of indole-3-acetic acid (IAA), pharmaceutical compositions thereof, and methods of modulating and treating diseases associated with IAA. The recombinant bacteria are capable of producing indole-3-acetic acid in low-oxygen environments, e.g., the gut. Thus, the recombinant bacteria and pharmaceutical compositions comprising those bacteria are non-pathogenic, and can be used in order to treat and/or prevent conditions associated with metabolic diseases, including obesity, diabetes, and liver diseases.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including, but not limited to”.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with the term “or”, “at least one of” or “one or more of” the elements in a list, unless context clearly indicates otherwise.

The term “about” is used herein to mean within the typical ranges of tolerances in the art, e.g., acceptable variation in time between doses, acceptable variation in dosage unit amount. For example, “about” can be understood as within about 2 standard deviations from the mean. In certain embodiments, about means+10%. In certain embodiments, about means+5%. When about is present before a series of numbers or a range, it is understood that “about” can modify each of the numbers in the series or range.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

As used herein, the term “recombinant bacterial cell” or “recombinant bacterium” refers to a bacterial cell or bacteria that have been genetically modified from their native state. For instance, an engineered bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Engineered bacterial cells disclosed herein may comprise exogenous nucleotide sequences on plasmids. Alternatively, engineered bacterial cells may comprise exogenous nucleotide sequences stably incorporated into their chromosome.

A “programmed bacterial cell” or “programmed engineered bacterial cell” is an engineered bacterial cell that has been genetically modified from its native state to perform a specific function. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures. In one embodiment, the regulatory sequence comprises a promoter, e.g., an FNR responsive promoter.

As used herein, a “gene cassette” or “operon” refers to a functioning unit of DNA containing a set of linked genes under the control of a single promoter. The genes are transcribed together into an mRNA strand and then translated for expression. A gene cassette encoding a biosynthetic pathway refers to two or more genes that are required to produce a molecule, e.g., indole-3-acetic acid. In addition to encoding a set of genes capable of producing said molecule, the gene cassette or operon may also comprise additional transcription and translation elements, e.g., a ribosome binding site.

As used herein, the term “trp operon” refers to a group of genes that is used, or transcribed, together that codes for the components for production of tryptophan. The trp operon is present in many bacteria, but was first characterized in Escherichia coli. The operon is regulated so that when tryptophan is present in the environment, the genes for tryptophan synthesis are not expressed. Trp operon contains five structural genes: trpE, trpD, trpC, trpB, and trpA, which encode enzymatic parts of the pathway. It also contains a repressive regulator gene called trpR. trpR has a promoter where RNA polymerase binds and synthesizes mRNA for a regulatory protein. The protein that is synthesized by trpR then binds to the operator which then causes the transcription to be blocked. Within the operon's regulatory sequence, the operator is bound to the repressor protein in the presence of tryptophan (thereby preventing transcription) and is liberated in tryptophan's absence (thereby allowing transcription).

As used herein, the term “trpE” refers to the well-known gene encoding for the enzyme anthranilate synthase component 1 (TrpE). This enzyme is part of a heterotetrameric complex that catalyzes the two-step biosynthesis of anthranilate, an intermediate in the biosynthesis of L-tryptophan. In the first step, the glutamine-binding beta subunit (TrpG) of anthranilate synthase (AS) provides the glutamine amidotransferase activity which generates ammonia as a substrate that, along with chorismate, is used in the second step, catalyzed by TrpE to produce anthranilate. In the absence of TrpG, TrpE can synthesize anthranilate directly from chorismate and high concentrations of ammonia. In some embodiments, the gene trpE is functionally replaced or modified, e.g., codon optimized, for enhanced expression. In other embodiments, the gene trpE refers to a feedback resistant form of trpE, e.g., trpE^{fbr}.

As used herein, the term “trpD” refers to the well-known gene encoding for the enzyme anthranilate phosphoribosyltransferase (TrpD). TrpD is involved in step 2 of the subpathway that synthesizes L-tryptophan from chorismate. TrpD catalyzes the transfer of the phosphoribosyl group of 5-phosphorylribose-1-pyrophosphate to anthranilate to yield phosphoribosyl-anthranilate. In some embodiments, the gene trpD is functionally replaced or modified, e.g., codon optimized, for enhanced expression.

As used herein, the term “trpC” refers to the well-known gene encoding for the enzyme indole-3-glycerolphosphate synthetase, which is the TrpC domain of the enzyme tryptophan biosynthesis protein (TrpCF). TrpCF is a bifunctional enzyme that catalyzes two sequential steps (steps 3 and 4) of tryptophan biosynthetic pathway. The first reaction is catalyzed by the isomerase, coded by the TrpF domain, converting phosphoribosyl-anthranilate to carboxyphenylamino-deoxyribulose-5-phosphate. The second reaction is catalyzed by the synthase, coded by the TrpC domain, converting carboxyphenylamino-deoxyribulose-5-phosphate to indole-3-glycerol phosphate. In some embodiments, the gene trpC is functionally replaced or modified, e.g., codon optimized, for enhanced expression.

As used herein, the terms “trpA” and “trpB” refer to the well-known genes encoding for the alpha chain (TrpA) and the beta chain (TrpB) of the enzyme tryptophan synthase, respectively. TrpA is responsible for the conversion of indole-3-glycerol phosphate to indole, and TrpB is responsible for the synthesis of L-tryptophan from indole and L-serine. In some embodiments, the gene trpA is functionally replaced or modified, e.g., codon optimized, for enhanced expression. In some embodiments, the gene trpB is codon-optimized for enhanced expression.

As used herein, the term “aroG” refers to the well-known gene encoding for the enzyme phospho-2-dehydro-3-deoxyheptonate aldolase (AroG). This enzyme is involved in the first step of the subpathway that synthesizes chorismate from D-erythrose 4-phosphate and phosphoenolpyruvate. AroG catalyzes the stereospecific condensation of phosphoenolpyruvate and D-erythrose-4-phosphate giving rise to 3-deoxy-D-arabino-heptulosonate-7-phosphate. In some embodiments, the gene aroG is functionally replaced or modified, e.g., codon optimized, for enhanced expression. In other embodiments, the gene aroG refers to a feedback resistant form of aroG, e.g., aroG^{fbr}. In some embodiments, a ribosome binding site is added before the gene aroG^{fbr}.

As used herein, the term “trpDH” refers to the well-known gene encoding for the enzyme tryptophan dehydrogenase (trpDH). This enzyme catalyzes the conversion of L-tryptophan to indole-3-pyruvate. In some embodiments, the gene trpDH is functionally replaced or modified, e.g., codon optimized, for enhanced expression. In other embodiments, a ribosome binding site is added before the gene trpDH.

As used herein, the term “ipdC” refers to the well-known gene encoding for the enzyme indole-3-pyruvate decarboxylase (IpdC). This enzyme catalyzes the conversion of indole-3-pyruvate to indole-3-acetaldehyde. In some embodiments, the gene ipdC is functionally replaced or modified, e.g., codon optimized, for enhanced expression. In other embodiments, a ribosome binding site is added before the gene ipdC.

As used herein, the term “iad1” refers to the well-known gene encoding for the enzyme indole-3-acetaldehyde dehydrogenase (Iad1). This enzyme catalyzes the conversion of indole-3-acetaldehyde to indole-3-acetic acid. In some embodiments, the gene iad1 is functionally replaced or modified, e.g., codon optimized, for enhanced expression. In other embodiments, a ribosome binding site is added before the gene iad1.

An “indole-3-acetic acid (IAA) gene cassette,” “IAA biosynthesis gene cassette,” and “IAA operon” are used interchangeably to refer to a set of genes capable of producing IAA in a biosynthetic pathway. Unmodified bacteria that are capable of producing IAA via an endogenous IAA biosynthesis pathway include, but are not limited to, Clostridium propionicum, Megasphaera elsdenii, and Prevotella ruminicola. The recombinant bacteria may comprise IAA biosynthesis genes from a different species, strain, or substrain of bacteria, or a combination of IAA biosynthesis genes from different species, strains, and/or substrains of bacteria. In some embodiments, the IAA gene cassette may comprise, for example, one or more of the genes selected from the group consisting of trpE^{fbr}, trpD, trpC, trpB, trpA, aroG^{fbr}, trpDH, ipdC and iad1. In some embodiments, the IAA gene cassette comprises trpE^{fbr}, trpD, trpC, trpB, and trpA. In other embodiments, the IAA gene cassette comprises aroG^{fbr}, trpDH, ipdC and iad1. In some embodiments, the genes may be functionally replaced or modified, e.g., codon optimized, for enhanced expression. In other embodiments, one or more ribosome binding sites are added to one or more of the genes in the gene cassette.

As used herein, the term “ribosome binding site” or “RBS” refers to a sequence of nucleotides upstream of the start codon of an mRNA transcript that is responsible for the recruitment of a ribosome during the initiation of protein translation. In some embodiments, one or more ribosome binding sites are added to one or more of the genes in the gene cassette described herein for enhanced expression. In other embodiments, the sequence for ribosome binding site is optimized for enhanced expression.

As used herein, the term “trpR” refers to the well-known gene encoding for the enzyme tryptophan transcriptional repressor (TrpR). Within the trp operon's regulatory sequence, the repressor protein TrpR will bind the operator in the presence of tryptophan, thereby preventing transcription. In the absence of tryptophan, TrpR will be liberated from the operator, thus allowing transcription of the downstream genes. In some embodiments, the trpR gene within the recombinant bacteria may be deleted, mutated, or modified so as to diminish or obliterate its repressor function.

As used herein, the term “tnaA” refers to the well-known gene encoding for the enzyme tryptophanase (TnaA), also known as tryptophan indole-lyase. TnaA is involved in the first step of the subpathway that synthesizes indole and pyruvate from L-tryptophan. In some embodiments, the tnaA gene within the recombinant bacteria may be deleted, mutated, or modified so as to inhibit the production of indole from tryptophan.

As used herein, the term “operably linked” refers a nucleic acid sequence, e.g., a gene or gene cassette for producing a metabolite, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns. In some embodiments, each gene or gene cassette may be operably linked to a promoter that is induced under low-oxygen conditions.

A “directly inducible promoter” refers to a regulatory region, wherein the regulatory region is operably linked to a gene or a gene cassette encoding a biosynthetic pathway for producing a metabolite, e.g., IAA. In the presence of an inducer of said regulatory region, a metabolic molecule is expressed.

An “indirectly inducible promoter” refers to a regulatory system comprising two or more regulatory regions, for example, a first regulatory region that is operably linked to a gene encoding a first molecule, e.g., a transcription factor, which is capable of regulating a second regulatory region that is operably linked to a gene or a gene cassette encoding a biosynthetic pathway for producing a metabolite, e.g., IAA. In the presence of an inducer of the first regulatory region, the second regulatory region may be activated or repressed, thereby activating or repressing production of IAA. Both a directly inducible promoter and an indirectly inducible promoter are encompassed by “inducible promoter.”

“Exogenous environmental condition(s)” refers to setting(s) or circumstance(s) under which the promoter described above is directly or indirectly induced. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen or anaerobic conditions such as the environment of the mammalian gut. In some embodiments, exogenous environmental conditions are molecules or metabolites that are specific to the mammalian gut, e.g., IAA. In some embodiments, the gene or gene cassette for producing a therapeutic molecule is operably linked to an oxygen level-dependent promoter. Bacteria have evolved transcription factors that are capable of sensing oxygen levels. Different signaling pathways may be triggered by different oxygen levels and occur with different kinetics.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression. In some embodiments, the gene or gene cassette for producing a metabolite, e.g., IAA, is operably linked to an oxygen level-dependent regulatory region such that the metabolite is expressed in low-oxygen, microaerobic, or anaerobic conditions. For example, the oxygen level-dependent regulatory region is operably linked to an IAA gene cassette. In low oxygen conditions, the oxygen level-dependent regulatory region is activated by a corresponding oxygen level-sensing transcription factor, thereby driving expression of the IAA gene cassette.

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the recombinant bacteria comprise a gene cassette that is operably linked to a directly or indirectly inducible promoter that is not associated with said gene cassette in nature, e.g., a FNR-responsive promoter operably linked to a IAA gene cassette.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg}(BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. In some aspects, the microorganism is engineered (“engineered microorganism”) to produce one or more therapeutic molecules. In certain aspects, the microorganism is engineered to import and/or catabolize certain toxic metabolites, substrates, or other compounds from its environment, e.g., the gut. In certain aspects, the microorganism is engineered to synthesize certain beneficial metabolites, molecules, or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism.

In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stably maintained” or “stable” bacterium is used to refer to a bacterial host cell carrying non-native genetic material, e.g., an IAA gene cassette, which is incorporated into the host genome or propagated on a self-replicating extra-chromosomal plasmid, such that the non-native genetic material is retained, expressed, and/or propagated. The stable bacterium is capable of survival and/or growth in vitro, e.g., in medium, and/or in vivo, e.g., in the gut. For example, the stable bacterium may be a genetically modified bacterium comprising an IAA gene cassette, in which the plasmid or chromosome carrying the IAA gene cassette is stably maintained in the host cell, such that the gene cassette can be expressed in the host cell, and the host cell is capable of survival and/or growth in vitro and/or in vivo.

As used herein, “metabolic diseases” include, but are not limited to, liver disease; non-alcoholic fatty liver disease (NAFLD); non-alcoholic steatohepatitis (NASH); liver cirrhosis; obesity; type 1 diabetes; type 2 diabetes; metabolic syndrome; Bardet-Biedel syndrome; Prader-Willi syndrome; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.

Symptoms associated with the aforementioned diseases and conditions include, but are not limited to, one or more of weight gain, obesity, fatigue, hyperlipidemia, hyperphagia, hyperdipsia, polyphagia, polydipsia, polyuria, pain of the extremities, numbness of the extremities, blurry vision, nystagmus, hearing loss, cardiomyopathy, insulin resistance, light sensitivity, pulmonary disease, liver disease, liver cirrhosis, liver failure, kidney disease, kidney failure, seizures, hypogonadism, and infertility.

Metabolic diseases are associated with a variety of physiological changes, including but not limited to elevated glucose levels, elevated triglyceride levels, elevated cholesterol levels, insulin resistance, high blood pressure, hypogonadism, subfertility, infertility, abdominal obesity, pro-thrombotic conditions, and pro-inflammatory conditions.

As used herein, the term “modulate” and its cognates means to alter, regulate, or adjust positively or negatively a molecular or physiological readout, outcome, or process, to effect a change in said readout, outcome, or process as compared to a normal, average, wild-type, or baseline measurement. Thus, for example, “modulate” or “modulation” includes up-regulation and down-regulation. A non-limiting example of modulating a readout, outcome, or process is effecting a change or alteration in the normal or baseline functioning, activity, expression, or secretion of a biomolecule (e.g., a protein, enzyme, cytokine, growth factor, hormone, metabolite, short chain fatty acid, or other compound). Another non-limiting example of modulating a readout, outcome, or process is effecting a change in the amount or level of a biomolecule of interest, e.g., in the serum and/or the gut lumen. In another non-limiting example, modulating a readout, outcome, or process relates to a phenotypic change or alteration in one or more disease symptoms. Thus, “modulate” is used to refer to an increase, decrease, masking, altering, overriding or restoring the normal functioning, activity, or levels of a readout, outcome or process (e.g., biomolecule of interest, and/or molecular or physiological process, and/or a phenotypic change in one or more disease symptoms).

As used herein, the term “treat” and its cognates refer to an amelioration of a disease or disorder, or at least one discernible symptom thereof. In another embodiment, “treat” refers to an amelioration of at least one measurable physical parameter, not necessarily discernible by the patient. In another embodiment, “treat” refers to inhibiting the progression of a disease or disorder, either physically (e.g., stabilization of a discernible symptom), physiologically (e.g., stabilization of a physical parameter), or both. In another embodiment, “treat” refers to slowing the progression or reversing the progression of a disease or disorder. As used herein, “prevent” and its cognates refer to delaying the onset or reducing the risk of acquiring a given disease or disorder.

Those in need of treatment may include individuals already having a particular medical disorder, as well as those at risk of having, or who may ultimately acquire the disorder. The need for treatment is assessed, for example, by the presence of one or more risk factors associated with the development of a disorder, the presence or progression of a disorder, or likely receptiveness to treatment of a subject having the disorder. Treating metabolic diseases may encompass reducing or eliminating associated symptoms, e.g., weight gain, and does not necessarily encompass the elimination of the underlying disease or disorder, e.g., liver disease. Treating the diseases described herein may encompass increasing levels of IAA, decreasing levels of tryptophan, and decreasing levels of tryptamine, and does not necessarily encompass the elimination of the underlying disease.

As used herein a “pharmaceutical composition” refers to a preparation of recombinant bacteria with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., liver disease. A therapeutically effective amount may, for example, be sufficient to treat, prevent, reduce the severity, delay the onset, and/or reduce the risk of occurrence of one or more symptoms of a metabolic disease. A therapeutically effective amount, as well as a therapeutically effective frequency of administration, can be determined by methods known in the art and discussed below.

### II. Recombinant Bacteria

The recombinant bacteria disclosed herein comprise a gene or gene cassette for producing a non-native metabolic molecule, e.g., indole-3-acetic acid (IAA). In some embodiments, the recombinant bacteria comprise one or more gene(s) or gene cassette(s) which are capable of producing the metabolite, e.g., IAA.

The recombinant bacteria may express any suitable set of IAA biosynthesis genes (see, e.g., Table 1). In some embodiments, the recombinant bacteria comprise the tryptophan operon of E. coli. (Yanofsky, RNA, 2007, 13:1141-1154). In some embodiments, the recombinant bacteria comprise the tryptophan operon of B. subtilis. (Yanofsky, RNA, 2007, 13:1141-1154). In some embodiments, the recombinant bacteria comprise one or more sequences encoding trypE^{fbr}, trypD, trypC, trypB, and trpA genes. In some embodiments, the recombinant bacteria comprise one or more sequences encoding trypE^{fbr}, trypD, trypC, trypB, and trpA genes from E. Coli. In some embodiments, the recombinant bacteria comprise one or more sequence encoding trypE^{fbr}, trypD, trypC, trypB, and trpA genes from B. subtilis.

In some embodiments, the recombinant bacteria may further comprise one or more genes which encode one or more enzymes to produce the tryptophan precursor, chorismate. Thus, in some embodiments, the recombinant bacteria may further comprise one or more sequences encoding aroG^{fbr}, aroF, aroH, aroB, aroD, aroE, aroK, and aroC genes. In some embodiments, the recombinant bacteria comprise one or more gene sequences encoding one or more enzymes of the tryptophan biosynthetic pathway and one or more gene sequences encoding one or more enzymes of the chorismate biosynthetic pathway. In some embodiments, the recombinant bacteria comprise one or more sequences encoding trypE^{fbr}, trypD, trypC, trypB, and trpA genes and sequence encoding the aroG^{fbr} gene.

In other embodiments, the recombinant bacteria may further comprise one or more genes which encode one or more tryptophan pathway catabolic enzymes, e.g., tryptophan dehydrogenase (trpDH), indole-3-pyruvate decarboxylase (ipdC), and indole-3-acetaldehyde (iad1). In some embodiments, the recombinant bacteria comprise one or more gene sequences encoding one or more enzymes of the tryptophan biosynthetic pathway, one or more gene sequences encoding one or more enzymes of the chorismate biosynthetic pathway, and one or more gene sequences encoding one or more tryptophan pathway catabolic enzymes. In some embodiments, the recombinant bacteria comprise one or more sequences encoding trypE^{fbr}, trypD, trypC, trypB, and trpA genes, the aroG^{fbr} gene, and the trpDH, ipdC and iad1 genes.

In some embodiments, the gene cassette comprises trypE^{fbr}, trypD, trypC, trypB, and trpA genes. In other embodiments, the gene cassette comprises aroG^{fbr}, trpDH, ipdC and iad1 genes. The genes may be codon-optimized, and translational and transcriptional elements may be added. In some embodiments, the gene or gene cassette for producing a metabolic molecule, e.g., IAA, comprises additional transcription and translation elements, e.g., a ribosome binding site, to enhance expression of the metabolic molecule. One or more ribosome binding sites may be added within a given gene cassette. In some embodiments, a ribosome binding site is added before the aroG^{fbr} gene. In other embodiments, a ribosome binding site is added before the trpDH gene. In some embodiments, a ribosome binding site is added before the ipdC gene. In other embodiments, a ribosome binding site is added before the iad1 gene. In another embodiment, a ribosome binding site is added before each of the genes, e.g., the aroG^{fbr} gene, the trpDH gene, the ipdC gene and/or the iad1 gene. In some embodiments, different ribosome binding sites are added before different genes. In other embodiments, the same ribosome binding site is added before different genes.

The gene or gene cassette for producing a metabolic molecule, e.g., IAA, also comprises additional modifications on the genes, e.g., a feedback resistant form of a gene, e.g., aroG^{fbr} and/or trpE^{fbr}, to enhance expression of the metabolic molecule.

Table 1 lists the nucleic acid sequences of exemplary genes in the IAA biosynthesis gene cassette. Table 2 lists the polypeptide sequences expressed by exemplary IAA biosynthesis genes.

TABLE 1
IAA Cassette Sequences

| Gene | Sequence |
|---|---|
| trpE^{fbr} | ATGCAAACACAAAAACCGACTCTCGAACTGCTAACCTGCGAAGGCGCTTATCGC |
| SEQ ID NO: 1 | GACAACCCGACTGCGCTTTTTCACCAGTTGTGTGGGGATCGTCCGGCAACGCTG |
| | CTGCTGGAATTCGCAGATATCGACAGCAAAGATGATTTAAAAAGCCTGCTGCTG |
| | GTAGACAGTGCGCTGCGCATTACAGCATTAAGTGACACTGTCACAATCCAGGCG |
| | CTTTCCGGCAATGGAGAAGCCCTGTTGACACTACTGGATAACGCCTTGCCTGCG |
| | GGTGTGGAAAATGAACAATCACCAAACTGCCGCGTACTGCGCTTCCCGCCTGTC |
| | AGTCCACTGCTGGATGAAGACGCCCGCTTATGCTCCCTTTCGGTTTTTGACGCT |
| | TTCCGCTTATTACAGAATCTGTTGAATGTACCGAAGGAAGAACGAGAAGCAATG |
| | TTCTTCGGCGGCCTGTTCTCTTATGACCTTGTGGCGGGATTTGAAAATTTACCG |
| | CAACTGTCAGCGGAAAATAGCTGCCCTGATTTCTGTTTTTATCTCGCTGAAACG |
| | CTGATGGTGATTGACCATCAGAAAAAAAGCACTCGTATTCAGGCCAGCCTGTTT |
| | GCTCCGAATGAAGAAGAAAAACAACGTCTCACTGCTCGCCTGAACGAACTACGT |
| | CAGCAACTGACCGAAGCCGCGCCGCCGCTGCCGGTGGTTTCCGTGCCGCATATG |
| | CGTTGTGAATGTAACCAGAGCGATGAAGAGTTCGGTGGTGTAGTGCGTTTGTTG |
| | CAAAAAGCGATTCGCGCCGGAGAAATTTTCCAGGTGGTGCCATCTCGCCGTTTC |
| | TCTCTGCCCTGCCCGTCACCGCTGGCAGCCTATTACGTGCTGAAAAAGAGTAAT |
| | CCCAGCCCGTACATGTTTTTTATGCAGGATAATGATTTCACCCTGTTTGGCGCG |
| | TCGCCGGAAAGTTCGCTCAAGTATGACGCCACCAGCCGCCAGATTGAGATTTAC |
| | CCGATTGCCGGAACACGTCCACGCGGTCGTCGTGCCGATGGTTCGCTGGACAGA |
| | GACCTCGACAGCCGCATCGAACTGGAGATGCGTACCGATCATAAAGAGCTTTCT |
| | GAACATCTGATGCTGGTGGATCTCGCCCGTAATGACCTGGCACGCATTTGCACA |
| | CCCGGCAGCCGCTACGTCGCCGATCTCACCAAAGTTGACCGTTACTCTTACGTG |
| | ATGCACCTAGTCTCCCGCGTTGTTGGTGAGCTGCGCCACGATCTCGACGCCCTG |
| | CACGCTTACCGCGCCTGTATGAATATGGGGACGTTAAGCGGTGCACCGAAAGTA |
| | CGCGCTATGCAGTTAATTGCCGAAGCAGAAGGTCGTCGACGCGGCAGCTACGGC |
| | GGCGCGGTAGGTTATTTTACCGCGCATGGCGATCTCGACACCTGCATTGTGATC |
| | CGCTCGGCGCTGGTGGAAAACGGTATCGCCACCGTGCAAGCCGGTGCTGGCGTA |
| | GTCCTTGATTCTGTTCCGCAGTCGGAAGCCGACGAAACTCGTAATAAAGCCCGC |
| | GCTGTACTGCGCGCTATTGCCACCGCGCATCATGCACAGGAGACGTTC |
| trpB | ATGACAACATTACTTAACCCCTATTTTGGTGAGTTTGGCGGCATGTACGTGCCA |
| SEQ ID NO: 2 | CAAATCCTGATGCCTGCTCTGCGCCAGCTGGAAGAAGCTTTTGTCAGCGCGCAA |
| | AAAGATCCTGAATTTCAGGCTCAGTTCAACGACCTGCTGAAAAACTATGCCGGG |
| | CGTCCAACCGCGCTGACCAAATGCCAGAACATTACAGCCGGGACGAACACCACG |
| | CTGTATCTGAAGCGCGAAGATTTGCTGCACGGCGGCGCGCATAAAACTAACCAG |
| | GTGCTCGGTCAGGCTTTACTGGCGAAGCGGATGGGTAAAACTGAAATTATTGCC |
| | GAAACCGGTGCCGGTCAGCATGGCGTGGCGTCGGCCCTTGCCAGCGCCCTGCTC |
| | GGCCTGAAATGCCGAATTTATATGGGTGCCAAAGACGTTGAACGCCAGTCGCCC |
| | AACGTTTTCCGGATGCGCTTAATGGGTGCGGAAGTGATCCCGGTACATAGCGGT |
| | TCCGCGACCCTGAAAGATGCCTGTAATGAGGCGCTACGCGACTGGTCCGGCAGT |
| | TATGAAACCGCGCACTATATGCTGGGTACCGCAGCTGGCCCGCATCCTTACCCG |
| | ACCATTGTGCGTGAGTTTCAGCGGATGATTGGCGAAGAAACGAAAGCGCAGATT |
| | CTGGAAAGAGAAGGTCGCCTGCCGGATGCCGTTATCGCCTGTGTTGGCGGTGGT |
| | TCGAATGCCATCGGTATGTTTGCAGATTTCATCAACGAAACCGACGTCGGCCTG |
| | ATTGGTGTGGAGCCTGGCGGCCACGGTATCGAAACTGGCGAGCACGGCGCACCG |
| | TTAAAACATGGTCGCGTGGGCATCTATTTCGGTATGAAAGCGCCGATGATGCAA |
| | ACCGAAGACGGGCAAATTGAAGAGTCTTACTCCATTTCTGCCGGGCTGGATTTC |
| | CCGTCCGTCGGCCCGCAACATGCGTATCTCAACAGCACTGGACGCGCTGATTAC |
| | GTGTCTATTACCGACGATGAAGCCCTGGAAGCCTTTAAAACGCTTTGCCTGCAT |
| | GAAGGGATCATCCCGGCGCTGGAATCCTCCCACGCCCTGGCCCATGCGCTGAAA |
| | ATGATGCGCGAAAATCCGGAAAAAGAGCAGCTACTGGTGGTTAACCTTTCCGGT |
| | CGCGGCGATAAAGACATCTTCACCGTTCACGATATTTTGAAAGCACGAGGGGAA |
| | ATCTGA |
| trpC | ATGCAAACCGTTTTAGCGAAAATCGTCGCAGACAAGGCGATTTGGGTAGAAACC |
| SEQ ID NO: 3 | CGCAAAGAGCAGCAACCGCTGGCCAGTTTTCAGAATGAGGTTCAGCCGAGCACG |
| | CGACATTTTTATGATGCACTTCAGGGCGCACGCACGGCGTTTATTCTGGAGTGT |
| | AAAAAAGCGTCGCCGTCAAAAGGCGTGATCCGTGATGATTTCGATCCGGCACGC |
| | ATTGCCGCCATTTATAAACATTACGCTTCGGCAATTTCAGTGCTGACTGATGAG |
| | AAATATTTTCAGGGGAGCTTTGATTTCCTCCCCATCGTCAGCCAAATCGCCCCG |
| | CAGCCGATTTTATGTAAAGACTTCATTATCGATCCTTACCAGATCTATCTGGCG |
| | CGCTATTACCAGGCCGATGCCTGCTTATTAATGCTTTCAGTACTGGATGACGAA |
| | CAATATCGCCAGCTTGCAGCCGTCGCCCACAGTCTGGAGATGGGTGTGCTGACC |
| | GAAGTCAGTAATGAAGAGGAACTGGAGCGCGCCATTGCATTGGGGGCAAAGGTC |
| | GTTGGCATCAACAACCGCGATCTGCGCGATTTGTCGATTGATCTCAACCGTACC |
| | CGCGAGCTTGCGCCGAAACTGGGGCACAACGTGACGGTAATCAGCGAATCCGGC |
| | ATCAATACTTACGCTCAGGTGCGCGAGTTAAGCCACTTCGCTAACGGCTTTCTG |
| | ATTGGTTCGGCGTTGATGGCCCATGACGATTTGAACGCCGCCGTGCGTCGGGTG |
| | TTGCTGGGTGAGAATAAAGTATGTGGCCTGACACGTGGGCAAGATGCTAAAGCA |
| | GCTTATGACGCGGGCGCGATTTACGGTGGGTTGATTTTTGTTGCGACATCACCG |
| | CGTTGCGTCAACGTTGAACAGGCGCAGGAAGTGATGGCTGCAGCACCGTTGCAG |
| | TATGTTGGCGTGTTCCGCAATCACGATATTGCCGATGTGGCGGACAAAGCTAAG |
| | GTGTTATCGCTGGCGGCAGTGCAACTGCATGGTAATGAAGATCAGCTGTATATC |
| | GACAATCTGCGTGAGGCTCTGCCAGCACACGTCGCCATCTGGAAGGCTTTAAGT |
| | GTCGGTGAAACTCTTCCCGCGCGCGATTTTCAGCACATCGATAAATATGTATTC |
| | GACAACGGTCAGGGCGGGAGCGGACAACGTTTCGACTGGTCACTATTAAATGGT |
| | CAATCGCTTGGCAACGTTCTGCTGGCGGGGGGCTTAGGCGCAGATAACTGCGTG |
| | GAAGCGGCACAAACCGGCTGCGCCGGGCTTGATTTTAATTCTGCTGTAGAGTCG |
| | CAACCGGGTATCAAAGACGCACGTCTTTTGGCCTCGGTTTTCCAGACGCTGCGC |
| | GCATATTAA |
| trpD | ATGGCTGACATTCTGCTGCTCGATAATATCGACTCTTTTACGTACAACCTGGCA |
| SEQ ID NO: 4 | GATCAGTTGCGCAGCAATGGTCATAACGTGGTGATTTACCGCAACCATATTCCG |
| | GCGCAGACCTTAATTGAACGCCTGGCGACGATGAGCAATCCGGTGCTGATGCTT |
| | TCTCCTGGCCCCGGTGTGCCGAGCGAAGCCGGTTGTATGCCGGAACTCCTCACC |
| | CGCTTGCGTGGCAAGCTGCCAATTATTGGCATTTGCCTCGGACATCAGGCGATT |
| | GTCGAAGCTTACGGGGGCTATGTCGGTCAGGCGGGCGAAATTCTTCACGGTAAA |
| | GCGTCGAGCATTGAACATGACGGTCAGGCGATGTTTGCCGGATTAACAAACCCG |
| | CTGCCAGTGGCGCGTTATCACTCGCTGGTTGGCAGTAACATTCCGGCCGGTTTA |
| | ACCATCAACGCCCATTTTAATGGCATGGTGATGGCGGTGCGTCACGATGCAGAT |
| | CGCGTTTGTGGATTCCAGTTCCATCCGGAATCCATTCTTACTACCCAGGGCGCT |
| | CGCCTGCTGGAACAAACGCTGGCCTGGGCGCAGCAGAAACTAGAGCCAACCAAC |
| | ACGCTGCAACCGATTCTGGAAAAACTGTATCAGGCACAGACGCTTAGCCAACAA |
| | GAAAGCCACCAGCTGTTTTCAGCGGTGGTACGTGGCGAGCTGAAGCCGGAACAA |
| | CTGGCGGCGGCGCTGGTGAGCATGAAAATTCGCGGTGAACACCCGAACGAGATC |
| | GCCGGGGCAGCAACCGCGCTACTGGAAAACGCCGCGCCATTCCCGCGCCCGGAT |
| | TATCTGTTTGCCGATATCGTCGGTACTGGCGGTGACGGCAGCAACAGCATCAAT |
| | ATTTCTACCGCCAGTGCGTTTGTCGCCGCGGCCTGCGGGCTGAAAGTGGCGAAA |
| | CACGGCAACCGTAGCGTCTCCAGTAAATCCGGCTCGTCGGATCTGCTGGCGGCG |
| | TTCGGTATTAATCTTGATATGAACGCCGATAAATCGCGCCAGGCGCTGGATGAG |
| | TTAGGCGTCTGTTTCCTCTTTGCGCCGAAGTATCACACCGGATTCCGCCATGCG |
| | ATGCCGGTTCGCCAGCAACTGAAAACCCGCACTCTGTTCAACGTGCTGGGACCA |
| | TTGATTAACCCGGCGCATCCGCCGCTGGCGCTAATTGGTGTTTATAGTCCGGAA |
| | CTGGTGCTGCCGATTGCCGAAACCTTGCGCGTGCTGGGGTATCAACGCGCGGCA |
| | GTGGTGCACAGCGGCGGGATGGATGAAGTTTCATTACACGCGCCGACAATCGTT |
| | GCCGAACTACATGACGGCGAAATTAAGAGCTATCAATTGACCGCTGAAGATTTT |
| | GGCCTGACACCCTACCACCAGGAGCAATTGGCAGGCGGAACACCGGAAGAAAAC |
| | CGTGACATTTTAACACGCTTGTTACAAGGTAAAGGCGACGCCGCCCATGAAGCA |
| | GCCGTCGCGGCGAATGTCGCCATGTTAATGCGCCTGCATGGCCATGAAGATCTG |
| | CAAGCCAATGCGCAAACCGTTCTTGAGGTACTGCGCAGTGGTTCCGCTTACGAC |
| | AGAGTCACCGCACTGGCGGCACGAGGGTAA |
| trpA | ATGGAACGCTACGAATCTCTGTTTGCCCAGTTGAAGGAGCGCAAAGAAGGCGCA |
| SEQ ID NO: 5 | TTCGTTCCTTTCGTCACCCTCGGTGATCCGGGCATTGAGCAGTCGTTGAAAATT |
| | ATCGATACGCTAATTGAAGCCGGTGCTGACGCGCTGGAGTTAGGCATCCCCTTC |
| | TCCGACCCACTGGCGGATGGCCCGACGATTCAAAACGCCACACTGCGTGCTTTT |
| | GCGGCGGGAGTAACCCCGGCGCAGTGCTTTGAGATGCTGGCACTCATTCGCCAG |
| | AAGCACCCGACCATTCCCATCGGCCTTTTGATGTATGCCAACCTGGTGTTTAAC |
| | AAAGGCATTGATGAGTTTTATGCCGAGTGCGAGAAAGTCGGCGTCGATTCGGTG |
| | CTGGTTGCCGATGTGCCCGTGGAAGAGTCCGCGCCCTTCCGCCAGGCCGCGTTG |
| | CGTCATAATGTCGCACCTATCTTTATTTGCCCGCCGAATGCCGACGATGATTTG |
| | CTGCGCCAGATAGCCTCTTACGGTCGTGGTTACACCTATTTGCTGTCGCGAGCG |
| | GGCGTGACCGGCGCAGAAAACCGCGCCGCGTTACCCCTCAATCATCTGGTTGCG |
| | AAGCTGAAAGAGTACAACGCTGCGCCTCCATTGCAGGGATTTGGTATTTCCGCC |
| | CCGGATCAGGTAAAAGCCGCGATTGATGCAGGAGCTGCGGGCGCGATTTCTGGT |
| | TCGGCCATCGTTAAAATCATCGAGCAACATATTAATGAGCCAGAGAAAATGCTG |
| | GCGGCACTGAAAGCTTTTGTACAACCGATGAAAGCGGCGACGCGCAGTTAA |
| aroG^{fbr} | ATGAATTATCAGAACGACGATTTACGCATCAAAGAAATCAAAGAGTTACTTCCT |
| SEQ ID NO: 6 | CCTGTCGCATTGCTGGAAAAATTCCCCGCTACTGAAAATGCCGCGAATACGGTC |
| | GCCCATGCCCGAAAAGCGATCCATAAGATCCTGAAAGGTAATGATGATCGCCTG |
| | TTGGTGGTGATTGGCCCATGCTCAATTCATGATCCTGTCGCGGCTAAAGAGTAT |
| | GCCACTCGCTTGCTGACGCTGCGTGAAGAGCTGCAAGATGAGCTGGAAATCGTG |
| | ATGCGCGTCTATTTTGAAAAGCCGCGTACTACGGTGGGCTGGAAAGGGCTGATT |
| | AACGATCCGCATATGGATAACAGCTTCCAGATCAACGACGGTCTGCGTATTGCC |
| | CGCAAATTGCTGCTCGATATTAACGACAGCGGTCTGCCAGCGGCGGGTGAATTC |
| | CTGGATATGATCACCCTACAATATCTCGCTGACCTGATGAGCTGGGGCGCAATT |
| | GGCGCACGTACCACCGAATCGCAGGTGCACCGCGAACTGGCGTCTGGTCTTTCT |
| | TGTCCGGTAGGTTTCAAAAATGGCACTGATGGTACGATTAAAGTGGCTATCGAT |
| | GCCATTAATGCCGCCGGTGCGCCGCACTGCTTCCTGTCCGTAACGAAATGGGGG |
| | CATTCGGCGATTGTGAATACCAGCGGTAACGGCGATTGCCATATCATTCTGCGC |
| | GGCGGTAAAGAGCCTAACTACAGCGCGAAGCACGTTGCTGAAGTGAAAGAAGGG |
| | CTGAACAAAGCAGGCCTGCCAGCGCAGGTGATGATCGATTTCAGCCATGCTAAC |
| | TCGTCAAAACAATTCAAAAAGCAGATGGATGTTTGTACTGAGGTTTGCCAGCAG |
| | ATTGCCGGTGGCGAAAAGGCCATTATTGGCGTGATGGTGGAAAGCCATCTGGTG |
| | GAAGGCAATCAGAGCCTCGAGAGCGGGGAACCGCTGGCCTACGGTAAGAGCATC |
| | ACCGATGCCTGCATTGGCTGGGATGATACCGATGCTCTGTTACGTCAACTGGCG |
| | AGTGCAGTAAAAGCGCGTCGCGGG |
| trpDH | ATGCTGTTATTCGAGACTGTGCGTGAAATGGGTCATGAGCAAGTCCTTTTCTGT |
| SEQ ID NO: 7 | CATAGCAAGAATCCCGAGATCAAGGCAATTATCGCAATCCACGATACCACCTTA |
| | GGACCGGCTATGGGCGCAACTCGTATCTTACCTTATATTAATGAGGAGGCTGCC |
| | CTGAAAGATGCATTACGTCTGTCCCGCGGAATGACTTACAAAGCAGCCTGCGCC |
| | AATATTCCCGCCGGGGGCGGCAAAGCCGTCATCATCGCTAACCCCGAAAACAAG |
| | ACCGATGACCTGTTACGCGCATACGGCCGTTTCGTGGACAGCTTGAACGGCCGT |
| | TTCATCACCGGGCAGGACGTTAACATTACGCCCGACGACGTTCGCACTATTTCG |
| | CAGGAGACTAAGTACGTGGTAGGCGTCTCAGAAAAGTCGGGAGGGCCGGCACCT |
| | ATCACCTCTCTGGGAGTATTTTTAGGCATCAAAGCCGCTGTAGAGTCGCGTTGG |
| | CAGTCTAAACGCCTGGATGGCATGAAAGTGGCGGTGCAAGGACTTGGGAACGTA |
| | GGAAAAAATCTTTGTCGCCATCTGCATGAACACGATGTACAACTTTTTGTGTCT |
| | GATGTCGATCCAATCAAGGCCGAGGAAGTAAAACGCTTATTCGGGGCGACTGTT |
| | GTCGAACCGACTGAAATCTATTCTTTAGATGTTGATATTTTTGCACCGTGTGCA |
| | CTTGGGGGTATTTTGAATAGCCATACCATCCCGTTCTTACAAGCCTCAATCATC |
| | GCAGGAGCAGCGAATAACCAGCTGGAGAACGAGCAACTTCATTCGCAGATGCTT |
| | GCGAAAAAGGGTATTCTTTACTCACCAGACTACGTTATCAATGCAGGAGGACTT |
| | ATCAATGTTTATAACGAAATGATCGGATATGACGAGGAAAAAGCATTCAAACAA |
| | GTTCATAACATCTACGATACGTTATTAGCGATTTTCGAAATTGCAAAAGAACAA |
| | GGTGTAACCACCAACGACGCGGCCCGTCGTTTAGCAGAGGATCGTATCAACAAC |
| | TCCAAACGCTCAAAGAGTAAAGCGATTGCGGCG |
| ipdC | ATGCGCACTCCATATTGTGTCGCTGATTACCTGTTGGACCGCTTGACTGATTGC |
| SEQ ID NO: 8 | GGTGCAGATCACCTGTTCGGTGTTCCAGGCGATTACAACCTGCAATTCCTGGAT |
| | CACGTTATCGACTCACCTGACATTTGCTGGGTCGGGTGTGCGAATGAGTTAAAC |
| | GCTTCCTATGCAGCAGACGGCTATGCACGCTGTAAGGGGTTTGCAGCATTGCTG |
| | ACCACTTTTGGTGTGGGAGAATTGAGTGCAATGAACGGGATCGCAGGATCCTAT |
| | GCTGAGCATGTGCCAGTGTTGCATATCGTTGGTGCACCAGGTACTGCTGCCCAA |
| | CAGCGTGGTGAGCTGTTGCATCACACCTTAGGTGATGGTGAGTTTCGCCACTTC |
| | TACCATATGAGCGAACCCATTACAGTCGCTCAAGCTGTTCTGACGGAGCAGAAT |
| | GCGTGCTACGAAATCGATCGTGTTCTTACCACCATGTTGCGTGAACGTCGTCCT |
| | GGGTATCTGATGTTGCCAGCAGATGTCGCGAAGAAAGCAGCCACACCACCAGTC |
| | AATGCACTGACACATAAGCAAGCTCACGCAGATTCTGCCTGTTTGAAGGCGTTT |
| | CGTGATGCAGCTGAGAACAAGCTTGCCATGTCGAAGCGTACGGCACTTCTTGCG |
| | GATTTCCTGGTTCTGCGTCATGGCCTGAAGCACGCACTTCAGAAATGGGTTAAG |
| | GAAGTGCCTATGGCACATGCCACGATGCTTATGGGTAAGGGTATCTTTGACGAA |
| | CGCCAAGCAGGATTCTATGGAACATACAGTGGAAGCGCATCCACTGGTGCTGTC |
| | AAGGAAGCCATCGAAGGAGCGGATACTGTGCTTTGCGTTGGGACCCGTTTCACA |
| | GACACGTTAACAGCTGGATTTACCCACCAATTAACGCCAGCTCAGACCATTGAA |
| | GTACAACCTCATGCAGCACGTGTTGGTGATGTCTGGTTCACTGGTATTCCCATG |
| | AACCAAGCTATCGAGACACTGGTCGAATTGTGCAAACAACATGTCCATGCAGGC |
| | TTGATGAGCTCGTCAAGTGGTGCTATTCCCTTCCCACAACCAGACGGCTCCTTG |
| | ACGCAGGAGAACTTCTGGCGTACCCTTCAAACTTTCATTCGTCCAGGTGACATC |
| | ATTCTTGCAGATCAAGGTACTTCGGCGTTTGGTGCTATTGACTTGCGTCTTCCA |
| | GCTGACGTGAACTTCATTGTCCAGCCTCTGTGGGGTTCGATCGGATACACTCTT |
| | GCTGCAGCATTCGGTGCACAAACAGCTTGTCCTAATCGTCGCGTAATCGTCCTG |
| | ACTGGTGACGGTGCAGCTCAGTTGACAATTCAGGAGTTAGGCAGCATGTTACGT |
| | GATAAGCAACACCCAATCATCTTGGTCCTGAACAACGAGGGTTACACTGTAGAG |
| | CGTGCCATCCATGGTGCAGAACAACGTTACAACGACATTGCGTTGTGGAATTGG |
| | ACCCATATTCCACAAGCTTTAAGCCTGGATCCGCAAAGTGAGTGCTGGCGTGTA |
| | TCCGAAGCTGAACAACTGGCAGATGTGCTGGAGAAGGTCGCACATCATGAACGC |
| | TTATCGTTGATCGAAGTCATGTTGCCTAAGGCAGATATTCCACCATTGTTAGGT |
| | GCTCTGACGAAAGCCTTGGAGGCGTGTAACAACGCC |
| iad1 | ATGCCCACTTTGAACCTTGATCTGCCGAACGGCATTAAATCGACGATTCAGGCT |
| SEQ ID NO: 9 | GATTTGTTCATCAATAACAAGTTCGTCCCAGCACTTGATGGCAAGACATTTGCG |
| | ACAATCAATCCATCGACAGGAAAAGAAATCGGCCAGGTGGCAGAGGCTTCCGCA |
| | AAGGACGTGGACCTGGCAGTTAAGGCTGCACGCGAAGCCTTCGAGACCACTTGG |
| | GGTGAGAACACACCAGGTGATGCACGTGGTCGCTTGTTGATCAAACTGGCTGAG |
| | CTTGTGGAGGCCAACATTGACGAGCTTGCAGCGATTGAATCCCTGGATAATGGT |
| | AAGGCTTTCTCAATTGCAAAGTCCTTCGATGTAGCTGCGGTTGCTGCCAACTTA |
| | CGTTACTATGGTGGCTGGGCTGATAAGAACCATGGTAAAGTAATGGAGGTCGAT |
| | ACAAAGCGTCTGAACTATACGCGTCACGAGCCCATCGGAGTCTGTGGACAGATC |
| | ATTCCATGGAACTTTCCACTGCTCATGTTTGCTTGGAAGTTGGGACCAGCCTTG |
| | GCTACTGGCAATACGATCGTCCTGAAGACTGCAGAGCAAACACCTCTGTCGGCT |
| | ATCAAGATGTGTGAATTGATCGTGGAGGCAGGATTTCCTCCAGGCGTAGTAAAT |
| | GTGATCTCGGGCTTTGGTCCTGTCGCAGGAGCAGCGATCTCACAGCACATGGAC |
| | ATCGATAAGATCGCTTTCACAGGAAGTACCCTGGTAGGACGTAACATCATGAAG |
| | GCAGCAGCATCCACGAACCTGAAGAAGGTCACGCTGGAATTGGGTGGAAAGAGT |
| | CCTAACATCATCTTCAAGGACGCGGATTTAGATCAAGCTGTGCGTTGGAGTGCG |
| | TTCGGAATCATGTTCAATCACGGCCAGTGCTGCTGTGCAGGGAGTCGCGTGTAT |
| | GTAGAGGAGTCCATCTATGATGCTTTCATGGAGAAGATGACGGCACATTGTAAG |
| | GCACTGCAAGTTGGTGATCCTTTCTCCGCAAATACGTTCCAGGGTCCGCAAGTT |
| | TCACAATTGCAATATGACCGTATCATGGAGTACATCGAATCTGGCAAGAAGGAC |
| | GCAAATCTGGCATTGGGAGGCGTCCGTAAGGGGAACGAAGGTTACTTCATCGAA |
| | CCGACAATCTTTACTGACGTCCCTCATGATGCAAAGATTGCTAAGGAAGAGATC |
| | TTTGGACCTGTAGTCGTTGTATCCAAGTTTAAGGATGAGAAAGATTTGATCCGC |
| | ATCGCCAATGATAGCATTTATGGCCTTGCAGCAGCAGTCTTCTCTCGCGACATT |
| | TCACGTGCGATCGAAACAGCTCATAAGCTGAAGGCAGGTACAGTATGGGTTAAC |
| | TGCTACAATCAGTTGATCCCACAAGTTCCATTCGGTGGGTACAAAGCGTCCGGG |
| | ATCGGTCGCGAATTAGGAGAATATGCGTTATCTAACTATACCAATATCAAGGCG |
| | GTCCATGTTAATCTGAGTCAACCTGCTCCCATT |
| RBS of aroG | ACCTATTGGTATAAGGAGGTAAATA |
| SEQ ID NO: 10 | |
| RBS of TRpDH | TAGGAGGTTTCTATCAAATTAAGGAGGTTATT |
| SEQ ID NO: 11 | |
| RBS of ipdC | GGTAGGACGATTGTCCGGTAGATTTAAGGAGGATTTTTTTTT |
| SEQ ID NO: 12 | |
| hRBS of iad1 | TCATTTTAGTCAATATCAGTATTGATTCCATACACGTAAGGAGGTTATTT |
| SEQ ID NO: 13 | |

In some embodiments, the recombinant bacteria comprise one or more nucleic acid sequence(s) of Table 1 (SEQ ID NO: 1-SEQ ID NO: 13) or a functional fragment thereof. In some embodiments, the recombinant bacteria comprise a nucleic acid sequence that, but for the redundancy of the genetic code, encodes the same polypeptide as one or more nucleic acids sequence(s) of Table 1 (SEQ ID NO: 1-SEQ ID NO: 13) or a functional fragment thereof. In some embodiments, recombinant bacteria comprise a nucleic acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to, comprises, or consists of the DNA sequence of one or more nucleic acid sequence(s) of Table 1 (SEQ ID NO: 1-SEQ ID NO: 13) or a functional fragment thereof, or a nucleic acid sequence that, but for the redundancy of the genetic code, encodes the same polypeptide as one or more nucleic acid sequence(s) of Table 1 (SEQ ID NO: 1-SEQ ID NO: 13) or a functional fragment thereof.

Table 2 lists exemplary polypeptide sequences, which may be encoded by the IAA production gene(s) or cassette(s) of the recombinant bacteria.

TABLE 2
Polypeptide Sequences for IAA Synthesis

| Enzyme | Amino Acid Sequence |
|---|---|
| TrpE^{fbr} | MQTQKPTLELLTCEGAYRDNPTALFHQLCGDRPATLLLEFADIDSKDDLKSLLL |
| SEQ ID NO: 14 | VDSALRITALSDTVTIQALSGNGEALLTLLDNALPAGVENEQSPNCRVLRFPPV |
| | SPLLDEDARLCSLSVFDAFRLLQNLLNVPKEEREAMFFGGLFSYDLVAGFENLP |
| | QLSAENSCPDFCFYLAETLMVIDHQKKSTRIQASLFAPNEEEKQRLTARLNELR |
| | QQLTEAAPPLPVVSVPHMRCECNQSDEEFGGVVRLLQKAIRAGEIFQVVPSRRF |
| | SLPCPSPLAAYYVLKKSNPSPYMFFMQDNDFTLFGASPESSLKYDATSRQIEIY |
| | PIAGTRPRGRRADGSLDRDLDSRIELEMRTDHKELSEHLMLVDLARNDLARICT |
| | PGSRYVADLTKVDRYSYVMHLVSRVVGELRHDLDALHAYRACMNMGTLSGAPKV |
| | RAMQLIAEAEGRRRGSYGGAVGYFTAHGDLDTCIVIRSALVENGIATVQAGAGV |
| | VLDSVPQSEADETRNKARAVLRAIATAHHAQETF |
| TrpB | MTTLLNPYFGEFGGMYVPQILMPALRQLEEAFVSAQKDPEFQAQFNDLLKNYAG |
| SEQ ID NO: 15 | RPTALTKCQNITAGTNTTLYLKREDLLHGGAHKTNQVLGQALLAKRMGKTEIIA |
| | ETGAGQHGVASALASALLGLKCRIYMGAKDVERQSPNVFRMRLMGAEVIPVHSG |
| | SATLKDACNEALRDWSGSYETAHYMLGTAAGPHPYPTIVREFQRMIGEETKAQI |
| | LEREGRLPDAVIACVGGGSNAIGMFADFINETDVGLIGVEPGGHGIETGEHGAP |
| | LKHGRVGIYFGMKAPMMQTEDGQIEESYSISAGLDFPSVGPQHAYLNSTGRADY |
| | VSITDDEALEAFKTLCLHEGIIPALESSHALAHALKMMRENPEKEQLLVVNLSG |
| | RGDKDIFTVHDILKARGEI |
| TrpC | MQTVLAKIVADKAIWVETRKEQQPLASFQNEVQPSTRHFYDALQGARTAFILEC |
| SEQ ID NO: 16 | KKASPSKGVIRDDFDPARIAAIYKHYASAISVLTDEKYFQGSFDFLPIVSQIAP |
| | QPILCKDFIIDPYQIYLARYYQADACLLMLSVLDDEQYRQLAAVAHSLEMGVLT |
| | EVSNEEELERAIALGAKVVGINNRDLRDLSIDLNRTRELAPKLGHNVTVISESG |
| | INTYAQVRELSHFANGFLIGSALMAHDDLNAAVRRVLLGENKVCGLTRGQDAKA |
| | AYDAGATYGGLIFVATSPRCVNVEQAQEVMAAAPLQYVGVFRNHDIADVADKAK |
| | VLSLAAVQLHGNEDQLYIDNLREALPAHVAIWKALSVGETLPARDFQHIDKYVF |
| | DNGQGGSGQRFDWSLLNGQSLGNVLLAGGLGADNCVEAAQTGCAGLDFNSAVES |
| | QPGIKDARLLASVFQTLRAY |
| TrpD | MADILLLDNIDSFTYNLADQLRSNGHNVVIYRNHIPAQTLIERLATMSNPVLML |
| SEQ ID NO: 17 | SPGPGVPSEAGCMPELLTRLRGKLPIIGICLGHQAIVEAYGGYVGQAGEILHGK |
| | ASSIEHDGQAMFAGLTNPLPVARYHSLVGSNIPAGLTINAHFNGMVMAVRHDAD |
| | RVCGFQFHPESILTTQGARLLEQTLAWAQQKLEPTNTLQPILEKLYQAQTLSQQ |
| | ESHQLFSAVVRGELKPEQLAAALVSMKIRGEHPNEIAGAATALLENAAPFPRPD |
| | YLFADIVGTGGDGSNSINISTASAFVAAACGLKVAKHGNRSVSSKSGSSDLLAA |
| | FGINLDMNADKSRQALDELGVCFLFAPKYHTGFRHAMPVRQQLKTRTLFNVLGP |
| | LINPAHPPLALIGVYSPELVLPIAETLRVLGYQRAAVVHSGGMDEVSLHAPTIV |
| | AELHDGEIKSYQLTAEDFGLTPYHQEQLAGGTPEENRDILTRLLQGKGDAAHEA |
| | AVAANVAMLMRLHGHEDLQANAQTVLEVLRSGSAYDRVTALAARG |
| TrpA | MERYESLFAQLKERKEGAFVPFVTLGDPGIEQSLKIIDTLIEAGADALELGIPF |
| SEQ ID NO: 18 | SDPLADGPTIQNATLRAFAAGVTPAQCFEMLALIRQKHPTIPIGLLMYANLVFN |
| | KGIDEFYAECEKVGVDSVLVADVPVEESAPFRQAALRHNVAPIFICPPNADDDL |
| | LRQIASYGRGYTYLLSRAGVTGAENRAALPLNHLVAKLKEYNAAPPLQGFGISA |
| | PDQVKAAIDAGAAGAISGSAIVKIIEQHINEPEKMLAALKAFVQPMKAATRS |
| AroG^{fbr} | MNYQNDDLRIKEIKELLPPVALLEKFPATENAANTVAHARKAIHKILKGNDDRL |
| SEQ ID NO: 19 | LVVIGPCSIHDPVAAKEYATRLLTLREELQDELEIVMRVYFEKPRTTVGWKGLI |
| | NDPHMDNSFQINDGLRIARKLLLDINDSGLPAAGEFLDMITLQYLADLMSWGAI |
| | GARTTESQVHRELASGLSCPVGFKNGTDGTIKVAIDAINAAGAPHCFLSVTKWG |
| | HSAIVNTSGNGDCHIILRGGKEPNYSAKHVAEVKEGLNKAGLPAQVMIDFSHAN |
| | SSKQFKKQMDVCTDVCQQIAGGEKAIIGVMVESHLVEGNQSLESGEPLAYGKSI |
| | TDACIGWDDTDALLRQLASAVKARRG |
| TrpDH | MLLFETVREMGHEQVLFCHSKNPEIKAIIAIHDTTLGPAMGATRILPYINEEAA |
| Nostoc | LKDALRLSRGMTYKAACANIPAGGGKAVIIANPENKTDDLLRAYGRFVDSLNGR |
| punctiforme | FITGQDVNITPDDVRTISQETKYVVGVSEKSGGPAPITSLGVFLGIKAAVESRW |
| NIES-2108 | QSKRLDGMKVAVQGLGNVGKNLCRHLHEHDVQLFVSDVDPIKAEEVKRLFGATV |
| SEQ ID NO: 20 | VEPTEIYSLDVDIFAPCALGGILNSHTIPFLQASIIAGAANNQLENEQLHSQML |
| | AKKGILYSPDYVINAGGLINVYNEMIGYDEEKAFKQVHNIYDTLLAIFEIAKEQ |
| | GVTTNDAARRLAEDRINNSKRSKSKAIAA |
| IpdC | MRTPYCVADYLLDRLTDCGADHLFGVPGDYNLQFLDHVIDSPDICWVGCANELN |
| Enterobacter | ASYAADGYARCKGFAALLTTFGVGELSAMNGIAGSYAEHVPVLHIVGAPGTAAQ |
| cloacae | QRGELLHHTLGDGEFRHFYHMSEPITVAQAVLTEQNACYEIDRVLTTMLRERRP |
| SEQ ID NO: 21 | GYLMLPADVAKKAATPPVNALTHKQAHADSACLKAFRDAAENKLAMSKRTALLA |
| | DFLVLRHGLKHALQKWVKEVPMAHATMLMGKGIFDERQAGFYGTYSGSASTGAV |
| | KEAIEGADTVLCVGTRFTDTLTAGFTHQLTPAQTIEVQPHAARVGDVWFTGIPM |
| | NQAIETLVELCKQHVHAGLMSSSSGAIPFPQPDGSLTQENFWRTLQTFIRPGDI |
| | ILADQGTSAFGAIDLRLPADVNFIVQPLWGSIGYTLAAAFGAQTACPNRRVIVL |
| | TGDGAAQLTIQELGSMLRDKQHPIILVLNNEGYTVERAIHGAEQRYNDIALWNW |
| | THIPQALSLDPQSECWRVSEAEQLADVLEKVAHHERLSLIEVMLPKADIPPLLG |
| | ALTKALEACNNA |
| Iad1 | MPTLNLDLPNGIKSTIQADLFINNKFVPALDGKTFATINPSTGKEIGQVAEASA |
| Ustilago maydis | KDVDLAVKAAREAFETTWGENTPGDARGRLLIKLAELVEANIDELAAIESLDNG |
| SEQ ID NO: 22 | KAFSIAKSFDVAAVAANLRYYGGWADKNHGKVMEVDTKRLNYTRHEPIGVCGQI |
| | IPWNFPLLMFAWKLGPALATGNTIVLKTAEQTPLSAIKMCELIVEAGFPPGVVN |
| | VISGFGPVAGAAISQHMDIDKIAFTGSTLVGRNIMKAAASTNLKKVTLELGGKS |
| | PNIIFKDADLDQAVRWSAFGIMFNHGQCCCAGSRVYVEESIYDAFMEKMTAHCK |
| | ALQVGDPFSANTFQGPQVSQLQYDRIMEYIESGKKDANLALGGVRKGNEGYFIE |
| | PTIFTDVPHDAKIAKEEIFGPVVVVSKFKDEKDLIRIANDSIYGLAAAVFSRDI |
| | SRAIETAHKLKAGTVWVNCYNQLIPQVPFGGYKASGIGRELGEYALSNYTNIKA |
| | VHVNLSQPAPI |

In some embodiments, the recombinant bacteria encode one or more polypeptide sequences of Table 2 (SEQ ID NO: 14-SEQ ID NO: 22) or a functional fragment or variant thereof. In some embodiments, recombinant bacteria comprise a polypeptide sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to, comprises, or consists of the polypeptide sequence of one or more polypeptide sequence of Table 2 (SEQ ID NO: 14-SEQ ID NO: 22) or a functional fragment thereof.

One of skill in the art would appreciate that additional genes and gene cassettes capable of producing the metabolite, e.g., IAA, are known in the art and may be expressed by the recombinant bacteria.

In some embodiments, the recombinant bacteria are capable of expressing any one or more of the gene or gene cassettes described herein and further comprise one or more antibiotic resistance circuits known in the art, e.g., ampicillin resistant.

In any of these embodiments, the gene encoding tryptophan repressor (trpR) may be deleted, mutated, or modified within the recombinant bacteria so as to diminish or obliterate its repressor function. Also, in any of these embodiments, the gene encoding tryptophanase or tryptophan indole-lyase (tnaA) may be deleted, mutated, or modified so as to inhibit the production of indole from tryptophan.

The gene or gene cassette for producing the metabolite may be expressed under the control of a promoter. The gene or gene cassette can be either directly or indirectly operably linked to a promoter. In some embodiments, the promoter is not operably linked with the gene or gene cassette in nature. In some embodiments, the gene or gene cassette is expressed under the control of a constitutive promoter. In another embodiment, the gene or gene cassette is expressed under the control of an inducible promoter. In some embodiments, the gene or gene cassette is expressed under the control of a promoter that is directly or indirectly induced by exogenous environmental conditions. In one embodiment, the gene or gene cassette is expressed under the control of a promoter that is directly or indirectly induced by low-oxygen or anaerobic conditions, wherein expression of the gene or gene cassette is activated under low-oxygen or anaerobic environments, such as the environment of the mammalian gut. In some embodiments, the gene or gene cassette is expressed under the control of an oxygen level-dependent promoter.

Examples of oxygen level-dependent transcription factors and corresponding promoters and/or regulatory regions include, but are not limited to, the fumarate and nitrate reductase regulator (FNR), the anaerobic arginine deiminiase and nitrate reductase regulator (ANR), and the dissimilatory nitrate respiration regulator (DNR). Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 3.

TABLE 3
Examples of transcription factors and
responsive genes and regulatory regions

| Transcription | Examples of responsive genes, |
|---|---|
| Factor | promoters, and/or regulatory regions: |
| FNR | nirB, ydfZ, pdhR, focA, ndH, hlyE, |
| | narK, narX, narG, yfiD, tdcD |
| ANR | arcDABC |
| DNR | norb, norC |

In certain embodiments, the bacterial cell comprises at least one gene, gene(s), or gene cassettes for producing the metabolite, e.g., IAA, which is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR-responsive promoter sequences are known in the art, and any suitable FNR-responsive promoter sequence(s) may be used in the recombinant bacteria. An exemplary FNR-responsive promoter sequences is provided in Table 4. Lowercase letters are ribosome binding sites.

TABLE 4
FNR Promoter Sequences

| FNR Responsive | |
|---|---|
| Promoter | Sequence |
| SEQ ID NO: 23 | AGTTGTTCTTATTGGTGGTGTTGCTTTATGGTTGCATCGTAGTAAATG |
| | GTTGTAACAAAAGCAATTTTTCCGGCTGTCTGTATACAAAAACGCCG |
| | CAAAGTTTGAGCGAAGTCAATAAACTCTCTACCCATTCAGGGCAATA |
| | TCTCTCTTggatccaaagtgaaCCCGC |

In one embodiment, the FNR responsive promoter comprises SEQ ID NO: 23.

In alternate embodiments, the recombinant bacteria comprising at least one gene, gene(s), or gene cassettes for producing the metabolite, e.g., IAA, is expressed under the control of an alternate oxygen level-dependent promoter, e.g., DNR (Trunk et al., Environ Microbiol. 2010; 12(6):1719-33) or ANR (Ray et al., FEMS Microbiol Lett. 1997; 156(2):227-32). In these embodiments, expression of the metabolite, e.g., IAA, is particularly activated in a low-oxygen or anaerobic environment, such as in the mammalian gut. In some embodiments, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene, gene(s), or gene cassettes for producing the metabolite, e.g., IAA, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., BMC Genomics. 2011; 12:51). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the recombinant bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, as compared to the wild-type promoter under the same conditions. In some embodiments, the recombinant bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., J Biol Chem. 2006; 281(44):33268-75).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene, gene(s), or gene cassettes for producing the metabolites, e.g., IAA, are present on different plasmids. In some embodiments, the gene encoding the oxygen level-sensing transcriptional regulator and the gene, gene(s), or gene cassettes for producing the metabolite, e.g., IAA, are present on different plasmids. In some embodiments, the gene encoding the oxygen level-sensing transcriptional regulator and the gene, gene(s), or gene cassettes for producing the metabolite, e.g., IAA, are present on the same plasmid.

In some embodiments, the gene encoding the oxygen level-sensing transcriptional regulator is present on a chromosome. In some embodiments, the gene encoding the oxygen level-sensing transcriptional regulator and the gene, gene(s), or gene cassettes for producing the metabolite, e.g., IAA, are present on different chromosomes. In some embodiments, the gene encoding the oxygen level-sensing transcriptional regulator and the gene, gene(s), or gene cassettes for producing the metabolite, e.g., IAA, are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene, gene(s), or gene cassettes for producing the metabolite, e.g., IAA. In some embodiments, expression of the transcriptional regulator is controlled by the same promoter that controls expression of the gene, gene(s), or gene cassettes for producing the metabolite, e.g., IAA. In some embodiments, the transcriptional regulator and the metabolite, e.g., IAA, are divergently transcribed from a promoter region.

In some embodiments, gene expression is further optimized by methods known in the art, e.g., by optimizing ribosomal binding sites and/or increasing mRNA stability.

In some embodiments, the gene or gene cassette for producing the metabolic and/or satiety molecule is expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism and assimilation of less favorable carbon sources when rapidly metabolizable carbohydrates, such as glucose, are present (Wu et al., Sci Rep. 2015; 5: 14921). This preference for glucose has been termed glucose repression, as well as carbon catabolite repression (Deutscher, Curr Opin Microbiol. 2008; 11(2):87-93; Görke and Stülke, Nature Reviews Microbiology, 2008, 6: 954). In some embodiments, expression of the gene or gene cassette is controlled by an oxygen level-dependent promoter fused to a CRP binding site. In some embodiments, expression of the gene or gene cassette is controlled by a FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the gene or gene cassette by recruiting RNA polymerase to the FNR promoter via direct protein-protein interactions. In the presence of glucose, cyclic AMP does not bind to CRP and gene transcription is repressed. In some embodiments, an oxygen level-dependent promoter (e.g., a FNR-responsive promoter) fused to a binding site for a transcriptional activator is used to ensure that the gene or gene cassette is not expressed under anaerobic conditions when sufficient amounts of glucose are present, e.g., by adding glucose to growth media in vitro.

In some embodiments, the gene or gene cassette for producing the metabolic and/or satiety molecule is expressed under the control of an oxygen level-dependent promoter operably linked to a detectable product, e.g., GFP, and can be used to screen for mutants. In some embodiments, the oxygen level-dependent promoter is mutagenized, and mutants are selected based upon the level of detectable product, e.g., by flow cytometry, fluorescence-activated cell sorting (FACS) when the detectable product fluoresces. In some embodiments, one or more transcription factor binding sites is mutagenized to increase or decrease binding. In alternate embodiments, the wild-type binding sites are left intact and the remainder of the regulatory region is subjected to mutagenesis. In some embodiments, the mutant promoter is inserted into the recombinant bacteria to increase expression of the metabolic and/or satiety effector molecule in low-oxygen conditions, as compared to wild type bacteria of the same subtype under the same conditions. In some embodiments, the oxygen level-sensing transcription factor and/or the oxygen level-dependent promoter is a synthetic, non-naturally occurring sequence.

In some embodiments, one or more of the genes in a gene cassette for producing a the metabolite, e.g., IAA, is mutated to increase expression of said molecule in low oxygen conditions, as compared to unmutated bacteria of the same subtype under the same conditions.

In one embodiment, the bacterial cell comprises a heterologous IAA gene cassette. In some embodiments, the disclosure provides a bacterial cell that comprises a heterologous IAA gene cassette operably linked to a first promoter. In one embodiment, the first promoter is an inducible promoter. In one embodiment, the bacterial cell comprises an IAA gene cassette from a different organism, e.g., a different species of bacteria. In another embodiment, the bacterial cell comprises more than one copy of a native gene encoding an IAA gene cassette. In yet another embodiment, the bacterial cell comprises at least one native gene encoding an IAA gene cassette, as well as at least one copy of an IAA gene cassette from a different organism, e.g., a different species of bacteria. In one embodiment, the bacterial cell comprises at least one, two, three, four, five, or six copies of a gene encoding an IAA gene cassette. In one embodiment, the bacterial cell comprises multiple copies of a gene or genes encoding an IAA gene cassette.

Multiple distinct IAA gene cassettes are known in the art. In some embodiments, an IAA gene cassette is encoded by a gene cassette derived from a bacterial species. In some embodiments, an IAA gene cassette is encoded by a gene cassette derived from a non-bacterial species. In some embodiments, an IAA gene cassette is encoded by a gene derived from a eukaryotic species, e.g., a fungi. In one embodiment, the gene encoding the IAA gene cassette is derived from an organism of the genus or species that includes, but is not limited to, Clostridium propionicum, Megasphaera elsdenii, or Prevotella ruminicola.

In one embodiment, the IAA gene cassette has been codon-optimized for use in the engineered bacterial cell. In one embodiment, the IAA gene cassette has been codon-optimized for use in Escherichia coli. In another embodiment, the IAA gene cassette has been codon-optimized for use in Lactococcus. When the IAA gene cassette is expressed in the engineered bacterial cells, the bacterial cells produce more IAA than unmodified bacteria of the same bacterial subtype under the same conditions (e.g., culture or environmental conditions). Thus, the recombinant bacteria comprising a heterologous IAA gene cassette may be used to generate IAA to treat liver disease, such as nonalcoholic steatohepatitis (NASH).

The present disclosure further comprises genes encoding functional fragments of IAA biosynthesis enzymes or functional variants of an IAA biosynthesis enzyme. As used herein, the term “functional fragment thereof” or “functional variant thereof” relates to an element having qualitative biological activity in common with the wild-type enzyme from which the fragment or variant was derived. For example, a functional fragment or a functional variant of a mutated IAA biosynthesis enzyme is one which retains essentially the same ability to synthesize IAA as the IAA biosynthesis enzyme from which the functional fragment or functional variant was derived. For example a polypeptide having IAA biosynthesis enzyme activity may be truncated at the N-terminus or C-terminus, and the retention of IAA biosynthesis enzyme activity assessed using assays known to those of skill in the art, including the exemplary assays provided herein. In one embodiment, the engineered bacterial cell comprises a heterologous gene encoding an IAA biosynthesis enzyme functional variant. In another embodiment, the engineered bacterial cell comprises a heterologous gene encoding an IAA biosynthesis enzyme functional fragment.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The present disclosure encompasses IAA biosynthesis enzymes comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

In some embodiments, an IAA biosynthesis enzyme is mutagenized; mutants exhibiting increased activity are selected; and the mutagenized gene encoding the IAA biosynthesis enzyme is isolated and inserted into the bacterial cell of the disclosure. The gene comprising the modifications described herein may be present on a plasmid or chromosome.

In one embodiment, the IAA biosynthesis gene cassette is from Clostridium spp. In one embodiment, the Clostridium spp. is Clostridium propionicum. In another embodiment, the IAA biosynthesis gene cassette is from a Megasphaera spp. In one embodiment, the Megasphaera spp. is Megasphaera elsdenii. In another embodiment, the IAA biosynthesis gene cassette is from Prevotella spp. In one embodiment, the Prevotella spp. is Prevotella ruminicola. Other IAA biosynthesis gene cassettes are well-known to one of ordinary skill in the art.

In some embodiments, the recombinant bacteria comprise the genes for IAA biosynthesis, e.g., trpE, trpD, trpC, trpB, trpA, aroG, trpDH, ipdC and iad1. The genes may be codon-optimized and/or modified, and translational and transcriptional elements may be added. In some embodiments, the recombinant bacteria comprise the genes for IAA biosynthesis, e.g., trpE^{fbr}, trpD, trpC, trpB, trpA, aroG^{fbr}, trpDH, ipdC and iad1.

In one embodiment, the trpE^{fbr} gene has at least about 80% identity with SEQ ID NO: 1. In another embodiment, the trpE^{fbr} gene has at least about 85% identity with SEQ ID NO: 1. In one embodiment, the trpE^{fbr} gene has at least about 90% identity with SEQ ID NO: 1. In one embodiment, the trpE^{fbr} gene has at least about 95% identity with SEQ ID NO: 1. In another embodiment, the trpE^{fbr} gene has at least about 96%, 97%, 98%, or 99% identity with SEQ ID NO: 1. Accordingly, in one embodiment, the trpE^{fbr} gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1. In another embodiment, the trpE^{fbr} gene comprises the sequence of SEQ ID NO: 1. In yet another embodiment the trpE^{fbr} gene consists of the sequence of SEQ ID NO: 1.

In one embodiment, the trpD gene has at least about 80% identity with SEQ ID NO: 2. In another embodiment, the trpD gene has at least about 85% identity with SEQ ID NO: 2. In one embodiment, the trpD gene has at least about 90% identity with SEQ ID NO: 2. In one embodiment, the trpD gene has at least about 95% identity with SEQ ID NO: 2. In another embodiment, the trpD gene has at least about 96%, 97%, 98%, or 99% identity with SEQ ID NO: 2. Accordingly, in one embodiment, the trpD gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 2. In another embodiment, the trpD gene comprises the sequence of SEQ ID NO: 2. In yet another embodiment the trpD gene consists of the sequence of SEQ ID NO: 2.

In one embodiment, the trpC gene has at least about 80% identity with SEQ ID NO: 3. In another embodiment, the trpC gene has at least about 85% identity with SEQ ID NO: 3. In one embodiment, the trpC gene has at least about 90% identity with SEQ ID NO: 3. In one embodiment, the trpC gene has at least about 95% identity with SEQ ID NO: 3. In another embodiment, the trpC gene has at least about 96%, 97%, 98%, or 99% identity with SEQ ID NO: 3. Accordingly, in one embodiment, the trpC gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 3. In another embodiment, the trpC gene comprises the sequence of SEQ ID NO: 3. In yet another embodiment the trpC gene consists of the sequence of SEQ ID NO: 3.

In one embodiment, the trpB gene has at least about 80% identity with SEQ ID NO: 4. In another embodiment, the trpB gene has at least about 85% identity with SEQ ID NO: 4. In one embodiment, the trpB gene has at least about 90% identity with SEQ ID NO: 4. In one embodiment, the trpB gene has at least about 95% identity with SEQ ID NO: 4. In another embodiment, the trpB gene has at least about 96%, 97%, 98%, or 99% identity with SEQ ID NO: 4. Accordingly, in one embodiment, the trpB gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 4. In another embodiment, the trpB gene comprises the sequence of SEQ ID NO: 4. In yet another embodiment the trpB gene consists of the sequence of SEQ ID NO: 4.

In one embodiment, the trpA gene has at least about 80% identity with SEQ ID NO: 5. In another embodiment, the trpA gene has at least about 85% identity with SEQ ID NO: 5. In one embodiment, the trpA gene has at least about 90% identity with SEQ ID NO: 5. In one embodiment, the trpA gene has at least about 95% identity with SEQ ID NO: 5. In another embodiment, the trpA gene has at least about 96%, 97%, 98%, or 99% identity with SEQ ID NO: 5. Accordingly, in one embodiment, the trpA gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 5. In another embodiment, the trpA gene comprises the sequence of SEQ ID NO: 5. In yet another embodiment the trpA gene consists of the sequence of SEQ ID NO: 5.

In one embodiment, the aroG^{fbr} gene has at least about 80% identity with SEQ ID NO: 6. In another embodiment, the aroG^{fbr} gene has at least about 85% identity with SEQ ID NO: 6. In one embodiment, the aroG^{fbr} gene has at least about 90% identity with SEQ ID NO: 6. In one embodiment, the aroG^{fbr} gene has at least about 95% identity with SEQ ID NO: 6. In another embodiment, the aroG^{fbr} gene has at least about 96%, 97%, 98%, or 99% identity with SEQ ID NO: 6. Accordingly, in one embodiment, the aroG^{fbr} gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 6. In another embodiment, the aroG^{fbr} gene comprises the sequence of SEQ ID NO: 6. In yet another embodiment the aroG^{fbr} gene consists of the sequence of SEQ ID NO: 6.

In one embodiment, the trpDH gene has at least about 80% identity with SEQ ID NO: 7. In another embodiment, the trpDHgene has at least about 85% identity with SEQ ID NO: 7. In one embodiment, the trpDH gene has at least about 90% identity with SEQ ID NO: 7. In one embodiment, the trpDHgene has at least about 95% identity with SEQ ID NO: 7. In another embodiment, the trpDHgene has at least about 96%, 97%, 98%, or 99% identity with SEQ ID NO: 7. Accordingly, in one embodiment, the trpDHgene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 7. In another embodiment, the trpDH gene comprises the sequence of SEQ ID NO: 7. In yet another embodiment the trpDH gene consists of the sequence of SEQ ID NO: 7.

In one embodiment, the ipdC gene has at least about 80% identity with SEQ ID NO: 8. In another embodiment, the ipdC gene has at least about 85% identity with SEQ ID NO: 8. In one embodiment, the ipdC gene has at least about 90% identity with SEQ ID NO: 8. In one embodiment, the ipdC gene has at least about 95% identity with SEQ ID NO: 8. In another embodiment, the ipdC gene has at least about 96%, 97%, 98%, or 99% identity with SEQ ID NO: 8. Accordingly, in one embodiment, the ipdC gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 8. In another embodiment, the ipdC gene comprises the sequence of SEQ ID NO: 8. In yet another embodiment the ipdC gene consists of the sequence of SEQ ID NO: 8.

In one embodiment, the iad1 gene has at least about 80% identity with SEQ ID NO: 9. In another embodiment, the iad1 gene has at least about 85% identity with SEQ ID NO: 9. In one embodiment, the iad1 gene has at least about 90% identity with SEQ ID NO: 9. In one embodiment, the iad1 gene has at least about 95% identity with SEQ ID NO: 9. In another embodiment, the iad1 gene has at least about 96%, 97%, 98%, or 99% identity with SEQ ID NO: 9. Accordingly, in one embodiment, the iad1 gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 9. In another embodiment, the iad1 gene comprises the sequence of SEQ ID NO: 9. In yet another embodiment the iad1 gene consists of the sequence of SEQ ID NO: 9.

In one embodiment, one or more polypeptides encoded by the IAA circuits and expressed by the recombinant bacteria have at least about 80% identity with one or more of SEQ ID NO: 14 through SEQ ID NO: 22. In another embodiment, one or more polypeptides encoded by the IAA circuits and expressed by the recombinant bacteria have at least about 85% identity with one or more of SEQ ID NO: 14 through SEQ ID NO: 22. In one embodiment, one or more polypeptides encoded by the IAA circuits and expressed by the recombinant bacteria have at least about 90% identity with one or more of SEQ ID NO: 14 through SEQ ID NO: 22. In one embodiment, one or more polypeptides encoded by the IAA circuits and expressed by the recombinant bacteria have at least about 95% identity with one or more of SEQ ID NO: 14 through SEQ ID NO: 22. In another embodiment, one or more polypeptides encoded by the IAA circuits and expressed by the recombinant bacteria have at least about 96%, 97%, 98%, or 99% identity with one or more of SEQ ID NO: 14 through SEQ ID NO: 22. Accordingly, in one embodiment, one or more polypeptides encoded by the IAA circuits and expressed by the recombinant bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 14 through SEQ ID NO: 22. In another embodiment, one or more polypeptides encoded by the IAA circuits and expressed by the recombinant bacteria one or more polypeptides encoded by the IAA circuits and expressed by the recombinant bacteria comprise the sequence of one or more of SEQ ID NO: 14 through SEQ ID NO: 22. In yet another embodiment one or more polypeptides encoded by the IAA circuits and expressed by the recombinant bacteria consist of or more of SEQ ID NO: 14 through SEQ ID NO: 22.

In some embodiments, one or more of the IAA biosynthesis genes is a synthetic IAA biosynthesis gene. In some embodiments, one or more of the IAA biosynthesis genes is an E. coli IAA biosynthesis gene. In some embodiments, one or more of the IAA biosynthesis genes is a C. glutamicum IAA biosynthesis gene. In some embodiments, one or more of the IAA biosynthesis genes is a C. propionicum IAA biosynthesis gene. In some embodiments, one or more of the IAA biosynthesis genes is a R. sphaeroides IAA biosynthesis gene. The IAA gene cassette may comprise genes for the aerobic biosynthesis of IAA and/or genes for the anaerobic or microaerobic biosynthesis of IAA.

In some embodiments, the recombinant bacteria comprise a combination of IAA biosynthesis genes from different species, strains, and/or substrains of bacteria, and are capable of producing IAA. In some embodiments, one or more of the IAA biosynthesis genes is functionally replaced, modified, and/or mutated in order to enhance stability and/or increase IAA production. In some embodiments, the recombinant bacteria are capable of expressing the IAA biosynthesis cassette and producing IAA in low-oxygen conditions, in the presence of certain molecules or metabolites, in the presence of molecules or metabolites associated with liver damage, inflammation or an inflammatory response, or in the presence of some other metabolite that may or may not be present in the gut.

The gene or gene cassette for producing the metabolite, e.g., IAA, may be present on a plasmid or bacterial chromosome. The gene or gene cassette for producing the metabolite, e.g., IAA, may be expressed on a high-copy plasmid, a low-copy plasmid, or a chromosome. In some embodiments, expression from the plasmid may be useful for increasing expression of the metabolite, e.g., IAA. In some embodiments, expression from the chromosome may be useful for increasing stability of expression of the metabolite, e.g., IAA. In some embodiments, the gene or gene cassette for producing the metabolite, e.g., IAA, is integrated into the bacterial chromosome at one or more integration sites in the recombinant bacteria. For example, one or more copies of the IAA biosynthesis gene cassette may be integrated into the bacterial chromosome. In some embodiments, the gene or gene cassette for producing the metabolite, e.g., IAA, is expressed from a plasmid in the recombinant bacteria.

In some embodiments, the bacteria are genetically engineered to include multiple mechanisms of action, e.g., circuits producing multiple copies of the same product (e.g., to enhance copy number) or circuits performing multiple different functions. In some embodiments, the gene or gene cassette for producing the metabolite, e.g., IAA, is inserted into the bacterial genome at one or more of the following insertion sites in E. coli Nissle: malE/K, araC/BAD, lacZ, thyA, malP/T. For example, the recombinant bacteria may include four copies of the gene, gene(s), or gene cassettes for producing the metabolite, e.g., IAA, inserted at four different insertion sites. Alternatively, the recombinant bacteria may include three copies of the gene, gene(s), or gene cassettes for producing the metabolite, e.g., IAA, inserted at three different insertion sites and three copies of the gene, gene(s), or gene cassettes for producing the metabolite, e.g., IAA, inserted at three different insertion sites. Any suitable insertion site may be used. The insertion site may be anywhere in the genome, e.g., in a gene required for survival and/or growth; in an active area of the genome, such as near the site of genome replication; and/or in between divergent promoters in order to reduce the risk of unintended transcription.

In addition, multiple copies of any gene, gene cassette, or regulatory region may be present in the bacterium, wherein one or more copies of the gene, gene cassette, or regulatory region may be mutated or otherwise altered as described herein. In some embodiments, the recombinant bacteria are engineered to comprise multiple copies of the same gene, gene cassette, or regulatory region in order to enhance copy number or to comprise multiple different components of a gene cassette performing multiple different functions.

In some embodiments, the recombinant bacteria are non-pathogenic bacteria. In some embodiments, the recombinant bacteria are commensal bacteria. In some embodiments, the recombinant bacteria are probiotic bacteria. In some embodiments, non-pathogenic bacteria are Gram-negative bacteria. In some embodiments, non-pathogenic bacteria are Gram-positive bacteria. In some embodiments, the recombinant bacteria are naturally pathogenic bacteria that are modified or mutated to reduce or eliminate pathogenicity. Exemplary bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the recombinant bacteria are selected from the group consisting of Bacteroidesfragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis.

In some embodiments, the recombinant bacteria are Escherichia coli strain Nissle 1917 (E. coli Nissle), a Gram-positive bacterium of the Enterobacteriaceae family that “has evolved into one of the best characterized probiotics” (Ukena et al., PLoS One. 2007 Dec. 12; 2(12):e1308). The strain is characterized by its complete harmlessness (Schultz, Inflamm Bowel Dis. 2008 July; 14(7):1012-8), and has GRAS (generally recognized as safe) status (Reister et al., J Biotechnol. 2014 Oct. 10; 187:106-7, emphasis added). Genomic sequencing confirmed that E. coli Nissle lacks prominent virulence factors (e.g., E. coli α-hemolysin, P-fimbrial adhesins) (Schultz, Inflamm Bowel Dis. 2008 July; 14(7):1012-8). In addition, it has been shown that E. coli Nissle does not carry pathogenic adhesion factors, does not produce any enterotoxins or cytotoxins, is not invasive, and is not uropathogenic (Sonnenborn et al., Microbial Ecology in Health and Disease. 2009; 21:122-158). As early as in 1917, E. coli Nissle was packaged into medicinal capsules, called Mutaflor, for therapeutic use. E. coli Nissle has since been used to treat ulcerative colitis in humans in vivo (Rembacken et al., Lancet. 1999 Aug. 21; 354(9179):635-9), to treat inflammatory bowel disease, Crohn's disease, and pouchitis in humans in vivo (Schultz, Inflamm Bowel Dis. 2008 July; 14(7):1012-8), and to inhibit enteroinvasive Salmonella, Legionella, Yersinia, and Shigella in vitro (Altenhoefer et al., FEMS Immunol Med Microbiol. 2004 Apr. 9; 40(3):223-9). It is commonly accepted that E. coli Nissle's “therapeutic efficacy and safety have convincingly been proven” (Ukena et al., PLoS One. 2007 Dec. 12; 2(12):e1308). In a recent study in non-human primates, Nissle was well tolerated by female cynomolgus monkeys after 28 days of daily NG dose administration at doses up to 1×1012 CFU/animal. No Nissle related mortality occurred and no Nissle related effects were identified upon clinical observation, body weight, and clinical pathology assessment (see, e.g., PCT/US16/34200).

One of ordinary skill in the art would appreciate that the genetic modifications disclosed herein may be adapted for other species, strains, and subtypes of bacteria.

Unmodified E. coli Nissle and the recombinant bacteria may be destroyed, e.g., by defense factors in the gut or blood serum (Sonnenborn et al., Microbial Ecology in Health and Disease. 2009; 21:122-158). Thus the recombinant bacteria may require continued administration. Residence time in vivo may be calculated for the recombinant bacteria.

Methods of measuring the level of metabolite, e.g., IAA, such as, mass spectrometry, gas chromatography, high-performance liquid chromatography (HPLC), are known in the art (see, e.g., Aboulnaga et al., J Bact. 2013; 195(16):3704-3713). In some embodiments, measuring the activity and/or expression of one or more gene products in the IAA gene cassette serves as a proxy measurement for IAA production. In some embodiments, the bacterial cells are harvested and lysed to measure IAA production. In alternate embodiments, IAA production is measured in the bacterial cell medium. In some embodiments, the recombinant bacteria produce at least about 1 μM, at least about 10 μM, at least about 100 μM, at least about 500 μM, at least about 1 mM, at least about 2 mM, at least about 3 mM, at least about 5 mM, at least about 10 mM, at least about 15 mM, at least about 20 mM, at least about 30 mM, at least about 40 mM, at least about 50 mM, at least about 60 mM, at least about 70 mM, at least about 80 mM, or at least about 90 mM of IAA in low-oxygen conditions.

In some embodiments, under conditions where the gene, gene(s), or gene cassettes for producing the metabolite, e.g., IAA, is expressed, the recombinant bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the metabolite as compared to unmodified bacteria of the same subtype under the same conditions. Certain unmodified bacteria will not have detectable levels of the metabolite, e.g., IAA. In embodiments using genetically modified forms of these bacteria, the metabolite, e.g., IAA, will be detectable under inducing conditions.

In some embodiments, the bacterium is capable of producing about 1 μM indole-3-acetic acid (IAA) to about 200 μM IAA. In some embodiments, the bacterium is capable of producing about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 110 μM, about 120 μM, about 130 μM, about 140 μM, about 150 μM, about 160 μM, about 170 μM, about 180 μM, about 190 μM or about 200 μM IAA. In some embodiments, the bacterium is capable of producing about 2-200 μM, about 5-150 μM, about 5-100 μM, about 10-100 μM, about 20-100 μM, about 20-80 μM, about 30-75 μM or about 5-80 μM IAA.

In some embodiments, the bacterium is capable of producing about 1 μM tryptophan to about 200 μM tryptophan. In some embodiments, the bacterium is capable of producing about 1 μM, about 2 μM, about 3 μM, about 4 μM, about 5 μM, about 6 μM, about 7 μM, about 8 μM, about 9 μM, about 1 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 110 μM, about 120 μM, about 130 μM, about 140 μM, about 150 μM, about 160 μM, about 170 μM, about 180 μM, about 190 μM or about 200 μM tryptophan. In some embodiments, the bacterium is capable of producing about 2-200 μM, about 5-150 μM, about 5-100 μM, about 10-100 μM, about 20-100 μM, about 20-80 μM, about 30-75 μM or about 5-80 μM tryptophan.

In some embodiments, quantitative PCR (qPCR) is used to amplify, detect, and/or quantify mRNA expression levels of the gene, gene(s), or gene cassettes for producing the metabolite, e.g., IAA. Primers may be designed and used to detect mRNA in a sample according to methods known in the art. In some embodiments, a fluorophore is added to a sample reaction mixture that may contain metabolite RNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the metabolite.

In some embodiments, quantitative PCR (qPCR) is used to amplify, detect, and/or quantify mRNA expression levels of the metabolite. Primers may be designed and used to detect mRNA in a sample according to methods known in the art. In some embodiments, a fluorophore is added to a sample reaction mixture that may contain metabolite mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the metabolite.

### III. Methods

Another aspect of the disclosure provides methods of treating diseases, e.g., metabolic diseases, e.g., obesity, diabetes and liver diseases, by administering to a subject in need thereof, a composition comprising the recombinant bacteria as described herein.

In some embodiments, the metabolic disease is selected from the group consisting of type 1 diabetes; type 2 diabetes; metabolic syndrome; Bardet-Biedel syndrome; Prader-Willi syndrome; non-alcoholic fatty liver disease; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome. In some embodiments, the disclosure provides methods for reducing, ameliorating, or eliminating one or more symptom(s) associated with these diseases, including but not limited to weight gain, obesity, fatigue, hyperlipidemia, hyperphagia, hyperdipsia, polyphagia, polydipsia, polyuria, pain of the extremities, numbness of the extremities, blurry vision, nystagmus, hearing loss, cardiomyopathy, insulin resistance, light sensitivity, pulmonary disease, liver disease, liver cirrhosis, liver failure, kidney disease, kidney failure, seizures, hypogonadism, and infertility. In some embodiments, the subject to be treated is a human patient.

The method may comprise preparing a pharmaceutical composition with at least one genetically engineered species, strain, or subtype of bacteria described herein, and administering the pharmaceutical composition to a subject in a therapeutically effective amount. In some embodiments, the recombinant bacteria are administered orally, e.g., in a liquid suspension. In some embodiments, the recombinant bacteria are lyophilized in a gel cap and administered orally. In some embodiments, the recombinant bacteria are administered via a feeding tube or gastric shunt. In some embodiments, the recombinant bacteria are administered rectally, e.g., by enema. In some embodiments, the recombinant bacteria are administered topically, intraintestinally, intrajejunally, intraduodenally, intraileally, and/or intracolically.

In certain embodiments, the recombinant bacteria described herein are administered to treat, manage, ameliorate, or prevent metabolic diseases in a subject. In some embodiments, the method of treating or ameliorating metabolic diseases allows one or more symptoms of the disease to improve by at least about 5%, 10%, 20%, 30%, 40%, 50%, 60%, 70%, 80%, 90%, 95%, or more as compared to levels in an untreated or control subject. In some embodiments, the symptom (e.g., obesity, insulin resistance) is measured by comparing measurements in a subject before and after administration of the recombinant bacteria. In some embodiments, the subject is a human subject.

Before, during, and after the administration of the recombinant bacteria in a subject, metabolites level, metabolic symptoms and manifestations may be measured in a biological sample, e.g., blood, serum, plasma, urine, fecal matter, peritoneal fluid, a sample collected from a tissue, such as liver, skeletal muscle, pancreas, epididymal fat, subcutaneous fat, and beige fat. The biological samples may be analyzed to measure symptoms and manifestations of metabolic diseases. Useful measurements include measures of lean mass, fat mass, body weight, food intake, GLP-1 levels, endotoxin levels, insulin levels, lipid levels, HbAlc levels, short-chain fatty acid levels, triglyceride levels, and nonesterified fatty acid levels. Useful assays include, but are not limited to, insulin tolerance tests, glucose tolerance tests, pyruvate tolerance tests, assays for intestinal permeability, and assays for glycaemia upon multiple fasting and refeeding time points. In some embodiments, the methods may include administration of the compositions to reduce metabolic symptoms and manifestations to baseline levels, e.g., levels comparable to those of a healthy control, in a subject. In some embodiments, the methods may include administration of the compositions to reduce metabolic symptoms and manifestations to undetectable levels in a subject, or to less than about 1%, 2%, 5%, 10%, 20%, 25%, 30%, 40%, 50%, 60%, 70%, 75%, or 80% of the subject's levels prior to treatment.

In certain embodiments, the recombinant bacteria are E. coli Nissle. The recombinant bacteria may be destroyed, e.g., by defense factors in the gut or blood serum (Sonnenborn et al., Microbial Ecology in Health and Disease. 2009; 21:122-158) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

The recombinant bacteria may be administered alone or in combination with one or more additional therapeutic agents, e.g., insulin. An important consideration in the selection of the one or more additional therapeutic agents is that the agent(s) should be compatible with the recombinant bacteria, e.g., the agent(s) must not kill the bacteria. The dosage of the recombinant bacteria and the frequency of administration may be selected based on the severity of the symptoms and the progression of the disorder. The appropriate therapeutically effective dose and/or frequency of administration can be selected by a treating clinician.

### Metabolic Diseases

### NASH

Non-alcoholic steatohepatitis (NASH) is a severe form of non-alcoholic fatty liver disease (NAFLD), where excess fat accumulation in the liver results in chronic inflammation and damage. Nonalcoholic fatty liver disease is a component of metabolic syndrome and a spectrum of liver disorders ranging from simple steatosis to nonalcoholic steatohepatitis (NASH). Simple liver steatosis is defined as a benign form of NAFLD with minimal risk of progression, in contrast to NASH, which tends to progress to cirrhosis in up to 20% of patients and can subsequently lead to liver failure or hepatocellular carcinoma. NASH affects approximately 3-5% of the population in America, especially in those identified as obese. NASH is characterized by such abnormalities as advanced lipotoxic metabolites, pro-inflammatory substrate, fibrosis, and increased hepatic lipid deposition. If left untreated, NASH can lead to cirrhosis, liver failure, and hepatocellular carcinoma.

Although patients diagnosed with alcoholic steatohepatitis demonstrate similar symptoms and liver damage, NASH develops in individuals who do not consume alcohol, and the underlying causes of NASH are unknown. Hepatic steatosis occurs when the amount of imported and synthesized lipids exceeds the export or catabolism in hepatocytes. An excess intake of fat or carbohydrate is the main cause of hepatic steatosis. NAFLD patients exhibit signs of liver inflammation and increased hepatic lipid accumulation. In addition, the development of NAFLD in obese individuals is closely associated with insulin resistance and other metabolic disorders and thus might be of clinical relevance). Therefore, possible causative factors include insulin resistance, cytokine imbalance (specifically, an increase in the tumor necrosis factor-alpha (TNF-α)/adiponectin ratio), and oxidative stress resulting from mitochondrial abnormalities.

Currently, there is no accepted approach to treating NASH. Therapy generally involves treating known risk factors such as correction of obesity through diet and exercise, treating hyperglycemia through diet and insulin, avoiding alcohol consumption, and avoiding unnecessary medication. In animal models, administration of butyrate has been shown to reduce hepatic steatosis, inflammation, and fat deposition (Jin et al., British J. Nutrition, 2015; 114(11):1745-1755; Endo et al., PLoS One, 2013; 8(5):e63388). Colonic IAA delivery has also been shown to reduce intrahepatocellular lipid content in NASH patients, including improvements in weight gain and intra-abdominal fat deposition (Chambers et al., Gut, 2015 November; 64(11):1744-54), and GLP-1 administration has been shown to reduce the degree of lipotoxic metabolites and pro-inflammatory substrates, both of which have been shown to speed NASH development, as well as reduce hepatic lipid deposition (Bernsmeier et al., PLoS One, 2014; 9(1):e87488; Armstrong et al., J. Hepatol., 2016 February; 64(2):399-408).

Studies have also suggested that rapid weight loss through bariatric surgery (e.g. gastric bypass) is effective in decreasing steatosis, hepatic inflammation, and fibrosis. Other treatments have involved using anti-diabetic medications such as metformin, rosiglitazone, and pioglitazone. Though inconclusive, the studies suggest that the medications stimulate insulin sensitivity in NASH patients, thus alleviating liver damage. In cases were NASH has resulted in advanced cirrhosis, the only treatment is a liver transplant.

The liver has both an arterial and venous blood supply, with the majority of hepatic blood flow coming from the gut via the portal vein. In NASH, the liver is exposed to potentially harmful substances derived from the gut (increased permeability and reduced intestinal integrity), including translocated bacteria, LPS and endotoxins as well as secreted cytokines. Translocated microbial products might contribute to the pathogenesis of fatty liver disease by several mechanisms, including stimulating pro-inflammatory and profibrotic pathways via a range of cytokines.

In some embodiments, the recombinant bacteria are useful for the prevention, treatment, and/or management of NAFLD and/or NASH. In some embodiments, the recombinant bacteria comprise circuits which reduce inflammation. In some embodiments the circuits stimulate insulin secretion and/or promote satiety.

In some embodiments, the recombinant bacteria comprise one or more gene cassettes for the production of metabolites, e.g., IAA. In some embodiments, the recombinant bacteria comprise one or more gene cassettes for the production of IAA for the treatment of NAFLD and/or NASH. In certain embodiments, the recombinant bacteria comprise one or more gene cassettes as described herein, which increase levels of IAA metabolites described herein in the patient, e.g., in the serum and/or in the gut.

In certain embodiments, the recombinant bacteria comprise one or more gene cassettes as described herein, which also modulate the levels of tryptophan and tryptophan metabolites, e.g., tryptamine, in a patient, e.g., in the serum and/or in the gut. In certain embodiments, the recombinant bacteria comprise one or more gene cassettes as described herein, which decrease tryptophan levels in the patient, e.g., in the serum and/or in the gut. In certain embodiments, the recombinant bacteria comprise one or more gene cassettes as described herein, which decrease tryptamine levels in the patient, e.g., in the serum and/or in the gut.

### Diabetes

Diabetes mellitus type 1 (also known as type 1 diabetes) is a form of diabetes mellitus that results from the autoimmune destruction of the insulin-producing beta cells in the pancreas. The subsequent lack of insulin leads to increased glucose in blood and urine. The classical symptoms are frequent urination, increased thirst, increased hunger, and weight loss. In some embodiments the recombinant bacteria described herein are useful in the treatment, prevention and/or management of diabetes mellitus.

Diabetes mellitus type 2 is a long term metabolic disorder that is characterized by high blood sugar, insulin resistance, and relative lack of insulin. Common symptoms include increased thirst, frequent urination, and unexplained weight loss. Symptoms may also include increased hunger, feeling tired, and sores that do not heal. Often symptoms come on slowly. Long-term complications from high blood sugar include heart disease, strokes, diabetic retinopathy which can result in blindness, kidney failure, and poor blood flow in the limbs which may lead to amputations.

Insulin resistance is generally regarded as a pathological condition in which cells fail to respond to the normal actions of the hormone insulin. Normally insulin produced when glucose enters the circulation after a meal triggers glucose uptake into cells. Under conditions of insulin resistance, the cells in the body are resistant to the insulin produced after a meal, preventing glucose uptake and leading to high blood sugar.

In some embodiments, the recombinant bacteria are useful for the prevention, treatment, and/or management of diabetes. In some embodiments, the recombinant bacteria comprise circuits which reduce inflammation. In some embodiments the circuits stimulate insulin secretion and/or promote satiety.

In some embodiments, the recombinant bacteria comprise one or more gene cassettes for the production of metabolites, e.g., IAA. In some embodiments, the recombinant bacteria comprise one or more gene cassettes for the production of IAA for the treatment of diabetes. In certain embodiments, the recombinant bacteria comprise one or more gene cassettes as described herein, which increase levels of IAA metabolites described herein in the patient, e.g., in the serum and/or in the gut.

In certain embodiments, the recombinant bacteria comprise one or more gene cassettes as described herein, which also modulate the levels of tryptophan and tryptophan metabolites, e.g., tryptamine, in a patient, e.g., in the serum and/or in the gut. In certain embodiments, the recombinant bacteria comprise one or more gene cassettes as described herein, which decrease tryptophan levels in the patient, e.g., in the serum and/or in the gut. In certain embodiments, the recombinant bacteria comprise one or more gene cassettes as described herein, which decrease tryptamine levels in the patient, e.g., in the serum and/or in the gut.

### Obesity

Obesity is a common, deadly, and costly disease in developed countries which impacts all age groups, race, and gender. Obesity can be classified as an inflammatory disease because it is associated with immune activation and a chronic, low-grade systemic inflammation. Endotoxemia, a process resulting from translocation of endotoxic compounds, e.g., lipopolysaccharides (LPS), of gram-negative intestinal bacteria. In the last decade, it has become evident that insulin resistance and type 2 diabetes are characterized by low-grade inflammation. In this respect, LPS trigger a low-grade inflammatory response, and the process of endotoxemia can therefore result in the development of insulin resistance and other metabolic disorders.

In some embodiments, the recombinant bacteria are useful for the prevention, treatment, and/or management of obesity. In some embodiments, the recombinant bacteria comprise circuits which reduce inflammation. In some embodiments the circuits stimulate insulin secretion and/or promote satiety.

In some embodiments, the recombinant bacteria comprise one or more gene cassettes for the production of metabolites, e.g., IAA. In some embodiments, the recombinant bacteria comprise one or more gene cassettes for the production of IAA for the treatment of obesity. In certain embodiments, the recombinant bacteria comprise one or more gene cassettes as described herein, which increase levels of IAA metabolites described herein in the patient, e.g., in the serum and/or in the gut.

In certain embodiments, the recombinant bacteria comprise one or more gene cassettes as described herein, which also modulate the levels of tryptophan and tryptophan metabolites, e.g., tryptamine, in a patient, e.g., in the serum and/or in the gut. In certain embodiments, the recombinant bacteria comprise one or more gene cassettes as described herein, which decrease tryptophan levels in the patient, e.g., in the serum and/or in the gut. In certain embodiments, the recombinant bacteria comprise one or more gene cassettes as described herein, which decrease tryptamine levels in the patient, e.g., in the serum and/or in the gut.

### Treatment In Vivo

The recombinant bacteria may be evaluated in vivo, e.g., in an animal model. Any suitable animal model of a metabolic disease may be used (see, e.g., Mizoguchi, Prog Mol Biol Transl Sci. 2012; 105:263-320). In some embodiments, the animal is a C57BL/6J mouse that is fed a high fat diet in order to induce obesity and T2DM-related symptoms such as hyperinsulinemia and hyperglycemia. In alternate embodiments, an animal harboring a genetic deficiency that causes a metabolic disease, e.g., a B6.BKS(D)-Lepr^{db/db} mouse, is used.

The recombinant bacteria are administered to the mice before, during, or after the onset of obesity and disease. Body weight, food intake, and blood plasma (e.g., triglyceride levels, insulin tolerance tests, glucose tolerance tests, pyruvate tolerance tests) may be assayed to determine the severity and amelioration of disease. Metabolism and physical activity may be measured in metabolic cages. Animals may be sacrificed to assay metabolic tissues such as liver, skeletal muscle, epididymal fat, subcutaneous fat, brown fat, pancreas, and brain, are collected for analysis of histology and gene expression.

The engineered bacteria may be evaluated in vivo, e.g., in an animal model for NASH. Any suitable animal model of a disease associated with NAFLD/NASH may be used. For example, the effects of liver steatosis and hepatic inflammation in an in vivo mouse model have been described (Jun Jin, et al., Brit. J. Nutrition, 2015; 114:145-1755). Briefly, female C57BL/6J mice can be fasted and fed either a standard liquid diet of carbohydrates, fat, and protein; or a liquid Western style diet fortified with fructose, fat, cholesterol, and a sodium butyrate supplement for six weeks. Body weight and plasma samples can be taken throughout the duration of the study. Upon conclusion of the study, the mice can be killed, and the liver and intestine can be removed and assayed.

An in vivo rat model of choline deficient/L-amino acid defined (CDAA) diet has also been described (Endo, et al., PLoS One, 8(5):e63388 (2013)). In this model, rats are fed the CDAA diet for eight weeks and then treated with a strain of Clostridium butyricum (MIYAIRI 588) two weeks after. The diet induces NAFLD/NASH symptoms such as liver steatosis, steatohepatitis, fibrosis, cirrhosis, and hepatocarcinogenesis. The rats are killed at 8, 16, and 50 weeks after completion of the diet regiments, and liver tissues removed and assayed.

Other models are known in the art, including a Lepob/Lepob and C57BL6 (B6) mouse model used to study the effects of high fat diet and GLP-1 administration within the NASH setting. See, for example, Trevaskis et al., Am. J. Physiology-Gastrointestinal and Liver Physiology, 2012; 302(8):G762-G772, and Takahashi et al., World J. Gastroenterol., 2012; 18(19):2300-2308, the entire contents of each of which are expressly incorporated herein by reference.

### IV. Pharmaceutical Compositions and Formulations

Pharmaceutical compositions comprising the recombinant bacteria may be used to treat, manage, ameliorate, and/or prevent a metabolic disease, e.g., obesity, diabetes and liver diseases. Pharmaceutical compositions comprising one or more recombinant bacteria, alone or in combination with prophylactic agents, therapeutic agents, and/or and pharmaceutically acceptable carriers are provided.

In certain embodiments, the pharmaceutical composition comprises one species, strain, or subtype of bacteria described herein that are engineered to treat, manage, ameliorate, and/or prevent a metabolic disease. In alternate embodiments, the pharmaceutical composition comprises two or more species, strains, and/or subtypes of bacteria described herein that are each engineered to treat, manage, ameliorate, and/or prevent a metabolic disease.

The pharmaceutical compositions may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The recombinant bacteria may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, immediate-release, pulsatile-release, delayed-release, or sustained release). Suitable dosage amounts for the recombinant bacteria may range from about 10⁵ to 10¹² bacteria, e.g., approximately 10⁵ bacteria, approximately 10⁶ bacteria, approximately 10⁷ bacteria, approximately 10⁸ bacteria, approximately 10⁹ bacteria, approximately 10¹⁰ bacteria, approximately 10¹¹ bacteria, or approximately 10¹¹ bacteria. The composition may be administered once or more daily, weekly, or monthly. The recombinant bacteria may be formulated into pharmaceutical compositions comprising one or more pharmaceutically acceptable carriers, thickeners, diluents, buffers, surface active agents, neutral or cationic lipids, lipid complexes, liposomes, penetration enhancers, carrier compounds, and other pharmaceutically acceptable carriers or agents.

The recombinant bacteria may be administered topically and formulated in the form of an ointment, cream, transdermal patch, lotion, gel, shampoo, spray, aerosol, solution, emulsion, or other form well-known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art.

The recombinant bacteria may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the recombinant bacteria are enterically coated for release into the gut or a particular region of the gut, for example, the small or large intestines. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionised at low pH, and therefore remain insoluble. But as the pH increases in the gastrointestinal tract, the acidic functional groups are capable of ionisation, and the polymer swells or becomes soluble in the intestinal fluid.

Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Coating polymers also may comprise one or more of, phthalate derivatives, CAT, HPMCAS, polyacrylic acid derivatives, copolymers comprising acrylic acid and at least one acrylic acid ester, Eudragit™ S (poly(methacrylic acid, methyl methacrylate)1:2); Eudragit L100™ S (poly(methacrylic acid, methyl methacrylate)1:1); Eudragit L30D™, (poly(methacrylic acid, ethyl acrylate)1:1); and (Eudragit L100-55) (poly(methacrylic acid, ethyl acrylate)1:1) (Eudragit™ L is an anionic polymer synthesized from methacrylic acid and methacrylic acid methyl ester), polymethyl methacrylate blended with acrylic acid and acrylic ester copolymers, alginic acid, ammonia alginate, sodium, potassium, magnesium or calcium alginate, vinyl acetate copolymers, polyvinyl acetate 30D (30% dispersion in water), a neutral methacrylic ester comprising poly(dimethylaminoethylacrylate) (“Eudragit E™), a copolymer of methylmethacrylate and ethylacrylate with trimethylammonioethyl methacrylate chloride, a copolymer of methylmethacrylate and ethylacrylate, Zein, shellac, gums, or polysaccharides, or a combination thereof.

Coating layers may also include polymers which contain Hydroxypropylmethylcellulose (HPMC), Hydroxypropylethylcellulose (HPEC), Hydroxypropylcellulose (HPC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), methylhydroxyethylcellulose (M H EC), hydrophobically modified hydroxyethylcellulose (NEXTON), carboxymethyl hydroxyethylcellulose (CMHEC), Methylcellulose, Ethylcellulose, water soluble vinyl acetate copolymers, gums, polysaccharides such as alginic acid and alginates such as ammonia alginate, sodium alginate, potassium alginate, acid phthalate of carbohydrates, amylose acetate phthalate, cellulose acetate phthalate (CAP), cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS).

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the recombinant bacteria.

In certain embodiments, the recombinant bacteria may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The recombinant bacteria may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The recombinant bacteria may be administered and formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, the disclosure provides pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

Dosage regimens may be adjusted to provide a therapeutic response. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The recombinant bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

Dosing can depend on several factors, including severity and responsiveness of the disease, route of administration, time course of treatment (days to months to years), and time to amelioration of the disease. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

### V. Kits

In certain aspects, the instant disclosure provides kits that include a pharmaceutical formulation including a recombinant bacterium for production of indole-3-acetic acid (IAA), and a package insert with instructions to perform any of the methods described herein.

In some embodiments, the kits include instructions for using the recombinant bacterium to treat a metabolic disease, e.g., obesity, diabetes or liver disease. The instructions will generally include information about the use of the recombinant bacterium to treat a metabolic disease, e.g., obesity, diabetes or liver disease. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

In some embodiments, the kit includes a pharmaceutical formulation including a recombinant bacterium for production of IAA, an additional therapeutic agent, and a package insert with instructions to perform any of the methods described herein.

The kit may be packaged in a number of different configurations such as one or more containers in a single box. The different components can be combined, e.g., according to instructions provided with the kit. The components can be combined according to a method described herein, e.g., to prepare and administer a pharmaceutical composition.

In some embodiments, the kit can comprise one or more containers with appropriate positive and negative controls or control samples, to be used as standard(s) for detection, calibration, or normalization.

The kit can further comprise a second container comprising a pharmaceutically-acceptable buffer, such as (sterile) phosphate-buffered saline, Ringer's solution, or dextrose solution; and other suitable additives such as penetration enhancers, carrier compounds and other pharmaceutically acceptable carriers or excipients, as described herein. It can further include other materials desirable from a commercial and user standpoint, including other buffers, diluents, filters, and package inserts with instructions for use. The kit can also include a drug delivery system such as liposomes, micelles, nanoparticles, and microspheres, as described herein. The kit can further include a delivery device, such as needles, syringes, pumps, and package inserts with instructions for use.

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

### Example 1. Production of Indole-3-Acetic Acid from Recombinant bacteria

To facilitate inducible production of indole-3-acetic acid (IAA) in Escherichia coli Nissle, a first IAA gene cassette comprising the following genes: trpE^{fbr}, trpB, trpC, trpD and trpA, as well as transcriptional and translational elements, were synthesized (Gen9, Cambridge, Mass.) and cloned into vector pSC101 (pSC101-amp-Pfnr-trpEfbrBCDA). The genes were codon-optimized for E. coli codon usage using Integrated DNA Technologies online codon optimization tool. A second clone was generated using a second IAA gene cassette comprising the genes: aroG^{fbr}, trpDH, ipdC and iad1 in a p15A vector. A ribosome binding site was added before each of the four genes (p15A-Pfnr-hRBS-aroGfbr-hRBS-trpDH-hRBS-ipdC-hRBS-iad1). Both gene cassettes were expressed under the control of a FNR-responsive promoter. Both constructs were transformed into E. coli Nissle and named as the 6741 strain. The presence of the IAA gene cassettes was verified by colony PCR.

| | | c | | | |
|---|---|---|---|---|---|
| SYN# | host | modification | plasmid 1 | plasmid 2 | induction |
| 6741 | 2126 | trpR, tnaA | pSC101-amp-Pfnr- | p15A-Pfnr-hRBS-aroG^{fbr}-hRBS- | O₂ minus |
| | | | trpE^{fbr}BCDA | trpDH-hRBS-ipdC-hRBS-iad1 | |

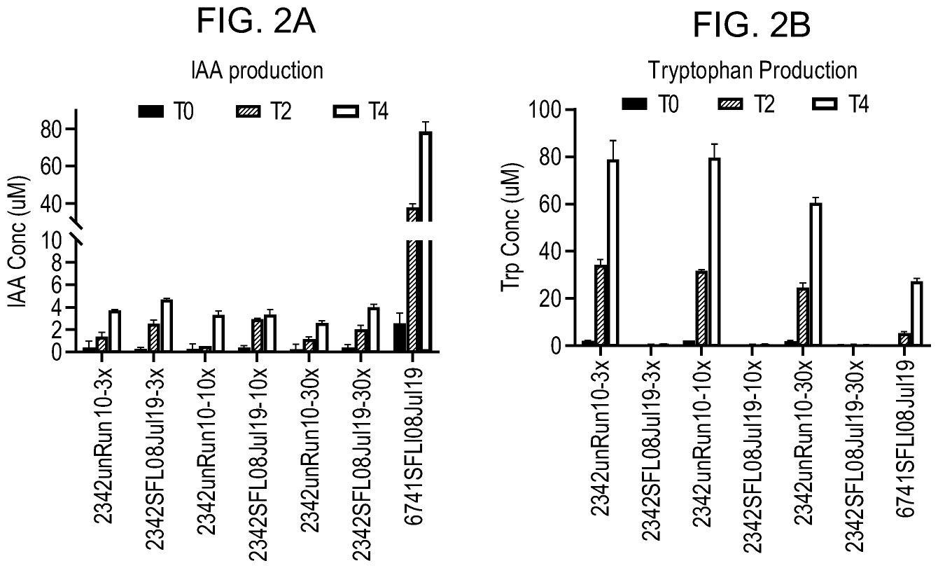

Production of IAA was assessed in E. coli Nissle strains containing the IAA cassettes. Cultures of E. coli Nissle transformed with the IAA cassettes were grown overnight in LB and then diluted 1:200 into 4 mL of M9 minimal medium containing 0.5% glucose. The cells were grown with shaking (250 rpm) for 4-6 h, and the inducible constructs were induced in LB at 37° C. for up to 4 hours in anaerobic conditions in a Coy anaerobic chamber (supplying 90% N2, 5% CO2, 5% H2, and 20 mM nitrate). One tube was removed at each time point (0, 2 and 4 hours) and analyzed for IAA concentration by LC-MS to confirm that IAA production in these recombinant strains can be achieved in a low-oxygen environment. As shown in FIGS. 2A and 2B, the level of IAA increased significantly in the 6471 strain, and the level of tryptophan decreased significantly overtime.

Process development Ambr screening fermentation was also carried out for cell cultures, and the effect of temperature on fermentation was examined. Specifically, a seed flask fermentation was started from a scraping of the frozen MCB culture in a cryo vial with an inoculum loop and added to FM1/25 g/L glucose media. Cultures of E coli Nissle transformed with the IAA cassettes were grown overnight in 10 mL of FM1 medium containing antibiotics in 50 mL baffled flasks at 37° C. with shaking at 250 RPM and diluted in 1:50 into 40 mL of FM1 containing antibiotics in 125 mL non-baffled flasks and incubated for 4 hours at 37° C. with shaking at 250 RPM. Cells were harvested by centrifugation, resuspended in PKU buffer and stored at −80° C. IAA production was performed by resuspending prepared cell pellet into 1.8 mL M9 minimal medium containing 50 mM MOPs and 2% glucose in a well of 96-deep well plates at OD 600=1. The plates were sealed with a breathable membrane and incubated at 37° C. incubator with shaking at 250 RPM or at 37° C. incubator in a Cory anaerobic chamber (supplying 90% N2, 5% CO2, 5% H2 and 20 mM Nitrate). Samples of 200 uL were taken at 0, 2 h and 4 h of incubation time and centrifuged for 8 min at 4000×g. The supernatants were analyzed for IAA concentrations by LC-MS. As shown in FIGS. 2A and 2B, the level of IAA increased significantly in the 6471 strain, and the level of tryptophan decreased significantly over time.

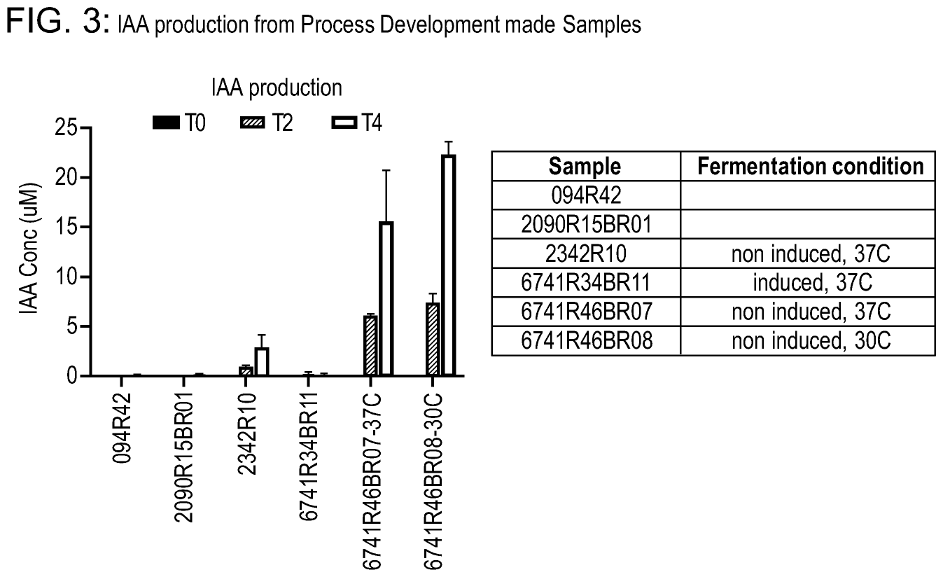

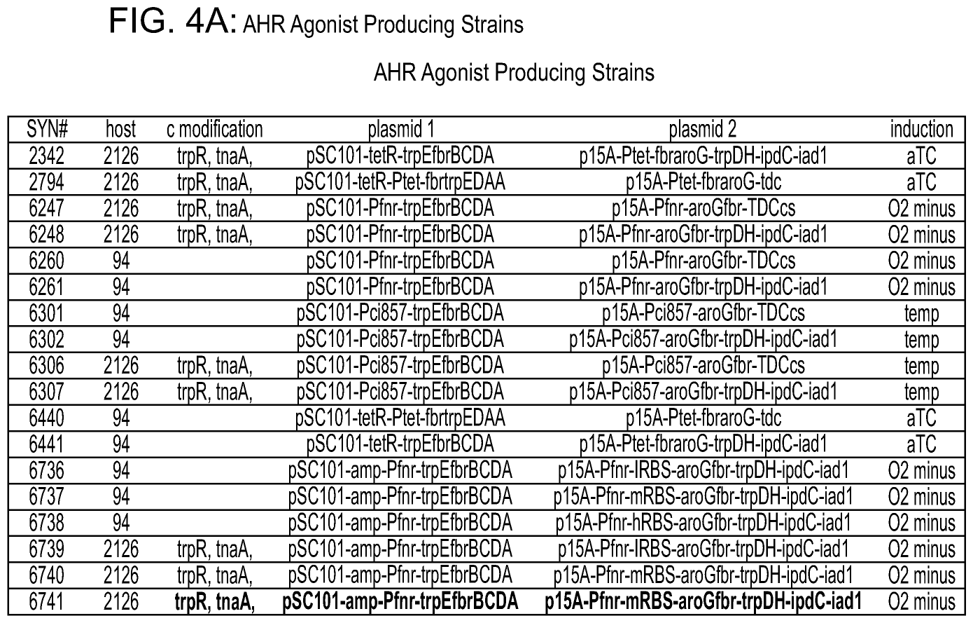

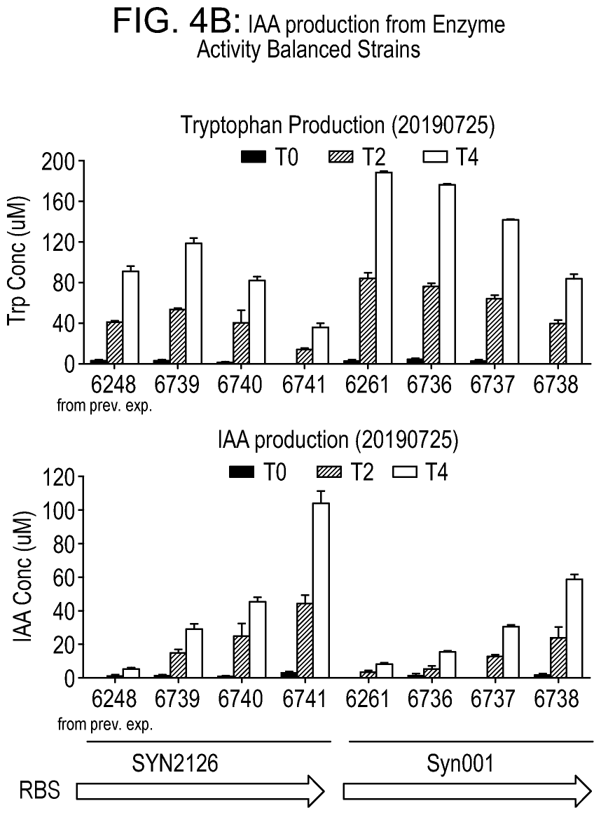

The culture continued to grow under the induction conditions for 2 hours before harvesting. They all were harvested at their targeted OD or induction endpoint and spun down by centrifuging culture broth for 15 min at 4500 RPM at 25 C. They were finally resuspended at a 10×concentration in PKU buffer, aliquoted and stored at −80° C. These cells were later thawed and ran in a Bioassay with flasks which were harvested at T=0, 2 h and 4 h. As demonstrated in FIG. 3, the IAA production level was higher under non-induced condition at 30° C. when compared to growth at 37° C. (both induced and non-induced conditions). Accordingly, in one embodiment, the bacteria are grown at a lower temperature, such as 30° C. to increase IAA production level.

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

We claim:

1. A recombinant bacterium comprising one or more gene cassettes for producing indole 3-acetic acid (IAA), wherein a first gene cassette comprises one or more gene sequences encoding a protein selected from the group consisting of TrpE, TrpB, TrpC, TrpD, TrpA, and a combination thereof, and
wherein the one or more gene cassettes are operably linked to a directly or indirectly inducible promoter that is not associated with the one or more gene sequences and is induced by exogenous environmental conditions.

2. The bacterium of claim 1, wherein the first gene cassette comprises gene sequences encoding TrpE, TrpB, TrpC, TrpD, and TrpA.

3. The bacterium of claim 1 or claim 2,
wherein the gene sequence encoding TrpE has at least 90%, 91%, 92%, 93%, 94%, 95%, 96%, 97%, 98%, 99% identity to, comprises, or consists of SEQ ID NO: 1,
wherein the gene sequence encoding TrpB has at least 90%, 91%, 92%, 93%, 94%, 95%, 96%, 97%, 98%, 99% identity to, comprises, or consists of SEQ ID NO: 2,
wherein the gene sequence encoding TrpC has at least 90%, 91%, 92%, 93%, 94%, 95%, 96%, 97%, 98%, 99% identity to, comprises, or consists of SEQ ID NO: 3,
wherein the gene sequence encoding TrpD has at least 90%, 91%, 92%, 93%, 94%, 95%, 96%, 97%, 98%, 99% identity to, comprises, or consists of SEQ ID NO: 4, and/or
wherein the gene sequence encoding TrpA has at least 90%, 91%, 92%, 93%, 94%, 95%, 96%, 97%, 98%, 99% identity to, comprises, or consists of SEQ ID NO: 5.

4. The bacterium of any one of the previous claims, further comprising a second gene cassette, wherein the second gene cassette comprises one or more sequences encoding a protein selected from the group consisting of AroG, TrpDH, IpdC, Iad1, and a combination thereof.

5. The bacterium of claim 4, wherein the second gene cassette comprises gene sequences encoding AroG, TrpDH, IpdC, and Iad1.

6. The bacterium of claim 4 or claim 5,
wherein the gene sequence encoding AroG has at least 90%, 91%, 92%, 93%, 94%, 95%, 96%, 97%, 98%, 99% identity to, comprises, or consists of SEQ ID NO: 6,
wherein the gene sequence encoding TrpDH has at least 90%, 91%, 92%, 93%, 94%, 95%, 96%, 97%, 98%, 99% identity to, comprises, or consists of SEQ ID NO: 7,
wherein the gene sequence encoding IpdC has at least 90%, 91%, 92%, 93%, 94%, 95%, 96%, 97%, 98%, 99% identity to, comprises, or consists of SEQ ID NO: 8, and/or
wherein the gene sequence encoding Iad1 has at least 90%, 91%, 92%, 93%, 94%, 95%, 96%, 97%, 98%, 99% identity to, comprises, or consists of SEQ ID NO: 9.

7. The bacterium of any one of claims 4-6, further comprising a ribosome binding site before the one or more gene sequences of the second gene cassette.

8. The bacterium of claim any one of the previous claims, wherein the bacterium further comprises a deletion or a mutation in a gene encoding tryptophan transcriptional repressor (TrpR) and/or a deletion or a mutation in a gene encoding tryptophanase (TnaA).

9. The bacterium of any one of the previous claims, wherein the promoter is directly or indirectly induced by low-oxygen or anaerobic conditions.

10. The bacterium of any one of the previous claims, wherein the promoter is an FNR-inducible promoter.

11. The bacterium of any one of the previous claims, wherein the one or more gene cassettes and operatively linked promoter are present on a plasmid in the bacterium.

12. The bacterium of any one of claims 1-10, wherein the one or more gene cassettes and operatively linked promoter are present on a chromosome in the bacterium.

13. The bacterium of any one of the previous claims, wherein the bacterium is a non-pathogenic bacterium.

14. The bacterium of any one of the previous claims, wherein the bacterium is a probiotic or a commensal bacterium.

15. The bacterium of claim any one of the previous claims, wherein the bacterium is selected from the group consisting of Bacteroides, Bifidobacterium, Clostridium, Escherichia, Lactobacillus, and Lactococcus.

16. The bacterium of claim 15, wherein the bacterium is Escherichia coli strain Nissle.

17. The bacterium of any one of the previous claims, wherein the bacterium is capable of producing about 1 μM IAA to about 200 μM IAA.

18. The bacterium of any one of the previous claims, wherein the bacterium is capable of producing about 1 μM IAA to about 200 μM tryptophan.

19. A recombinant bacterium comprising
one or more gene cassettes for producing indole 3-acetic acid (IAA), wherein a first gene cassette comprises one or more gene sequences encoding a protein selected from the group consisting of TrpE, TrpB, TrpC, TrpD, TrpA, and a combination thereof; and a second gene cassette, wherein the second gene cassette comprises one or more sequences encoding a protein selected from the group consisting of AroG, TrpDH, IpdC, Iad1, and a combination thereof; and
wherein the one or more gene cassettes are operably linked to a directly or indirectly inducible promoter that is not associated with the one or more gene sequences and is induced by exogenous environmental conditions.

20. A pharmaceutically acceptable composition comprising the bacterium of any one of the previous claims and a pharmaceutically acceptable carrier.

21. The pharmaceutically acceptable composition of claim 20, wherein the composition is formulated for oral administration.

22. A method of treating a disease in a subject in need thereof comprising the step of administering to the subject the pharmaceutical composition of claim 20 or claim 21.

23. The method of claim 22, wherein the disorder is selected from the group consisting of: liver disease; non-alcoholic fatty liver disease (NAFLD); non-alcoholic steatohepatitis (NASH); liver cirrhosis; obesity; type 1 diabetes; type 2 diabetes; metabolic syndrome; Bardet-Biedel syndrome; Prader-Willi syndrome; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.

24. The method of claim 22, wherein the subject has an increased level of IAA after the composition is administrated.

25. The method of claim 22, wherein the subject has a decreased level of tryptophan after the composition is administrated.

26. The method of claim 22, wherein the subject is a human.


SEQUENCE LISTING (23 sequences)

SEQ ID NO: 1 - DNA, 1560 bp, Artificial Sequence
  Description of Artificial Sequence Synthetic polynucleotide
atgcaaacacaaaaaccgactctcgaactgctaacctgcgaaggcgcttatcgcgacaacccgactgcgctttttcaccagttgtgtggggatcgtccggcaacgctgctgctggaattcgcagatatcgacagcaaagatgatttaaaaagcctgctgctggtagacagtgcgctgcgcattacagcattaagtgacactgtcacaatccaggcgctttccggcaatggagaagccctgttgacactactggataacgccttgcctgcgggtgtggaaaatgaacaatcaccaaactgccgcgtactgcgcttcccgcctgtcagtccactgctggatgaagacgcccgcttatgctccctttcggtttttgacgctttccgcttattacagaatctgttgaatgtaccgaaggaagaacgagaagcaatgttcttcggcggcctgttctcttatgaccttgtggcgggatttgaaaatttaccgcaactgtcagcggaaaatagctgccctgatttctgtttttatctcgctgaaacgctgatggtgattgaccatcagaaaaaaagcactcgtattcaggccagcctgtttgctccgaatgaagaagaaaaacaacgtctcactgctcgcctgaacgaactacgtcagcaactgaccgaagccgcgccgccgctgccggtggtttccgtgccgcatatgcgttgtgaatgtaaccagagcgatgaagagttcggtggtgtagtgcgtttgttgcaaaaagcgattcgcgccggagaaattttccaggtggtgccatctcgccgtttctctctgccctgcccgtcaccgctggcagcctattacgtgctgaaaaagagtaatcccagcccgtacatgttttttatgcaggataatgatttcaccctgtttggcgcgtcgccggaaagttcgctcaagtatgacgccaccagccgccagattgagatttacccgattgccggaacacgtccacgcggtcgtcgtgccgatggttcgctggacagagacctcgacagccgcatcgaactggagatgcgtaccgatcataaagagctttctgaacatctgatgctggtggatctcgcccgtaatgacctggcacgcatttgcacacccggcagccgctacgtcgccgatctcaccaaagttgaccgttactcttacgtgatgcacctagtctcccgcgttgttggtgagctgcgccacgatctcgacgccctgcacgcttaccgcgcctgtatgaatatggggacgttaagcggtgcaccgaaagtacgcgctatgcagttaattgccgaagcagaaggtcgtcgacgcggcagctacggcggcgcggtaggttattttaccgcgcatggcgatctcgacacctgcattgtgatccgctcggcgctggtggaaaacggtatcgccaccgtgcaagccggtgctggcgtagtccttgattctgttccgcagtcggaagccgacgaaactcgtaataaagcccgcgctgtactgcgcgctattgccaccgcgcatcatgcacaggagacgttc

SEQ ID NO: 2 - DNA, 1194 bp, Artificial Sequence
  Description of Artificial Sequence Synthetic polynucleotide
atgacaacattacttaacccctattttggtgagtttggcggcatgtacgtgccacaaatcctgatgcctgctctgcgccagctggaagaagcttttgtcagcgcgcaaaaagatcctgaatttcaggctcagttcaacgacctgctgaaaaactatgccgggcgtccaaccgcgctgaccaaatgccagaacattacagccgggacgaacaccacgctgtatctgaagcgcgaagatttgctgcacggcggcgcgcataaaactaaccaggtgctcggtcaggctttactggcgaagcggatgggtaaaactgaaattattgccgaaaccggtgccggtcagcatggcgtggcgtcggcccttgccagcgccctgctcggcctgaaatgccgaatttatatgggtgccaaagacgttgaacgccagtcgcccaacgttttccggatgcgcttaatgggtgcggaagtgatcccggtacatagcggttccgcgaccctgaaagatgcctgtaatgaggcgctacgcgactggtccggcagttatgaaaccgcgcactatatgctgggtaccgcagctggcccgcatccttacccgaccattgtgcgtgagtttcagcggatgattggcgaagaaacgaaagcgcagattctggaaagagaaggtcgcctgccggatgccgttatcgcctgtgttggcggtggttcgaatgccatcggtatgtttgcagatttcatcaacgaaaccgacgtcggcctgattggtgtggagcctggcggccacggtatcgaaactggcgagcacggcgcaccgttaaaacatggtcgcgtgggcatctatttcggtatgaaagcgccgatgatgcaaaccgaagacgggcaaattgaagagtcttactccatttctgccgggctggatttcccgtccgtcggcccgcaacatgcgtatctcaacagcactggacgcgctgattacgtgtctattaccgacgatgaagccctggaagcctttaaaacgctttgcctgcatgaagggatcatcccggcgctggaatcctcccacgccctggcccatgcgctgaaaatgatgcgcgaaaatccggaaaaagagcagctactggtggttaacctttccggtcgcggcgataaagacatcttcaccgttcacgatattttgaaagcacgaggggaaatctga

SEQ ID NO: 3 - DNA, 1359 bp, Artificial Sequence
  Description of Artificial Sequence Synthetic polynucleotide
atgcaaaccgttttagcgaaaatcgtcgcagacaaggcgatttgggtagaaacccgcaaagagcagcaaccgctggccagttttcagaatgaggttcagccgagcacgcgacatttttatgatgcacttcagggcgcacgcacggcgtttattctggagtgtaaaaaagcgtcgccgtcaaaaggcgtgatccgtgatgatttcgatccggcacgcattgccgccatttataaacattacgcttcggcaatttcagtgctgactgatgagaaatattttcaggggagctttgatttcctccccatcgtcagccaaatcgccccgcagccgattttatgtaaagacttcattatcgatccttaccagatctatctggcgcgctattaccaggccgatgcctgcttattaatgctttcagtactggatgacgaacaatatcgccagcttgcagccgtcgcccacagtctggagatgggtgtgctgaccgaagtcagtaatgaagaggaactggagcgcgccattgcattgggggcaaaggtcgttggcatcaacaaccgcgatctgcgcgatttgtcgattgatctcaaccgtacccgcgagcttgcgccgaaactggggcacaacgtgacggtaatcagcgaatccggcatcaatacttacgctcaggtgcgcgagttaagccacttcgctaacggctttctgattggttcggcgttgatggcccatgacgatttgaacgccgccgtgcgtcgggtgttgctgggtgagaataaagtatgtggcctgacacgtgggcaagatgctaaagcagcttatgacgcgggcgcgatttacggtgggttgatttttgttgcgacatcaccgcgttgcgtcaacgttgaacaggcgcaggaagtgatggctgcagcaccgttgcagtatgttggcgtgttccgcaatcacgatattgccgatgtggcggacaaagctaaggtgttatcgctggcggcagtgcaactgcatggtaatgaagatcagctgtatatcgacaatctgcgtgaggctctgccagcacacgtcgccatctggaaggctttaagtgtcggtgaaactcttcccgcgcgcgattttcagcacatcgataaatatgtattcgacaacggtcagggcgggagcggacaacgtttcgactggtcactattaaatggtcaatcgcttggcaacgttctgctggcggggggcttaggcgcagataactgcgtggaagcggcacaaaccggctgcgccgggcttgattttaattctgctgtagagtcgcaaccgggtatcaaagacgcacgtcttttggcctcggttttccagacgctgcgcgcatattaa

SEQ ID NO: 4 - DNA, 1596 bp, Artificial Sequence
  Description of Artificial Sequence Synthetic polynucleotide
atggctgacattctgctgctcgataatatcgactcttttacgtacaacctggcagatcagttgcgcagcaatggtcataacgtggtgatttaccgcaaccatattccggcgcagaccttaattgaacgcctggcgacgatgagcaatccggtgctgatgctttctcctggccccggtgtgccgagcgaagccggttgtatgccggaactcctcacccgcttgcgtggcaagctgccaattattggcatttgcctcggacatcaggcgattgtcgaagcttacgggggctatgtcggtcaggcgggcgaaattcttcacggtaaagcgtcgagcattgaacatgacggtcaggcgatgtttgccggattaacaaacccgctgccagtggcgcgttatcactcgctggttggcagtaacattccggccggtttaaccatcaacgcccattttaatggcatggtgatggcggtgcgtcacgatgcagatcgcgtttgtggattccagttccatccggaatccattcttactacccagggcgctcgcctgctggaacaaacgctggcctgggcgcagcagaaactagagccaaccaacacgctgcaaccgattctggaaaaactgtatcaggcacagacgcttagccaacaagaaagccaccagctgttttcagcggtggtacgtggcgagctgaagccggaacaactggcggcggcgctggtgagcatgaaaattcgcggtgaacacccgaacgagatcgccggggcagcaaccgcgctactggaaaacgccgcgccattcccgcgcccggattatctgtttgccgatatcgtcggtactggcggtgacggcagcaacagcatcaatatttctaccgccagtgcgtttgtcgccgcggcctgcgggctgaaagtggcgaaacacggcaaccgtagcgtctccagtaaatccggctcgtcggatctgctggcggcgttcggtattaatcttgatatgaacgccgataaatcgcgccaggcgctggatgagttaggcgtctgtttcctctttgcgccgaagtatcacaccggattccgccatgcgatgccggttcgccagcaactgaaaacccgcactctgttcaacgtgctgggaccattgattaacccggcgcatccgccgctggcgctaattggtgtttatagtccggaactggtgctgccgattgccgaaaccttgcgcgtgctggggtatcaacgcgcggcagtggtgcacagcggcgggatggatgaagtttcattacacgcgccgacaatcgttgccgaactacatgacggcgaaattaagagctatcaattgaccgctgaagattttggcctgacaccctaccaccaggagcaattggcaggcggaacaccggaagaaaaccgtgacattttaacacgcttgttacaaggtaaaggcgacgccgcccatgaagcagccgtcgcggcgaatgtcgccatgttaatgcgcctgcatggccatgaagatctgcaagccaatgcgcaaaccgttcttgaggtactgcgcagtggttccgcttacgacagagtcaccgcactggcggcacgagggtaa

SEQ ID NO: 5 - DNA, 807 bp, Artificial Sequence
  Description of Artificial Sequence Synthetic polynucleotide
atggaacgctacgaatctctgtttgcccagttgaaggagcgcaaagaaggcgcattcgttcctttcgtcaccctcggtgatccgggcattgagcagtcgttgaaaattatcgatacgctaattgaagccggtgctgacgcgctggagttaggcatccccttctccgacccactggcggatggcccgacgattcaaaacgccacactgcgtgcttttgcggcgggagtaaccccggcgcagtgctttgagatgctggcactcattcgccagaagcacccgaccattcccatcggccttttgatgtatgccaacctggtgtttaacaaaggcattgatgagttttatgccgagtgcgagaaagtcggcgtcgattcggtgctggttgccgatgtgcccgtggaagagtccgcgcccttccgccaggccgcgttgcgtcataatgtcgcacctatctttatttgcccgccgaatgccgacgatgatttgctgcgccagatagcctcttacggtcgtggttacacctatttgctgtcgcgagcgggcgtgaccggcgcagaaaaccgcgccgcgttacccctcaatcatctggttgcgaagctgaaagagtacaacgctgcgcctccattgcagggatttggtatttccgccccggatcaggtaaaagccgcgattgatgcaggagctgcgggcgcgatttctggttcggccatcgttaaaatcatcgagcaacatattaatgagccagagaaaatgctggcggcactgaaagcttttgtacaaccgatgaaagcggcgacgcgcagttaa

SEQ ID NO: 6 - DNA, 1050 bp, Artificial Sequence
  Description of Artificial Sequence Synthetic polynucleotide
atgaattatcagaacgacgatttacgcatcaaagaaatcaaagagttacttcctcctgtcgcattgctggaaaaattccccgctactgaaaatgccgcgaatacggtcgcccatgcccgaaaagcgatccataagatcctgaaaggtaatgatgatcgcctgttggtggtgattggcccatgctcaattcatgatcctgtcgcggctaaagagtatgccactcgcttgctgacgctgcgtgaagagctgcaagatgagctggaaatcgtgatgcgcgtctattttgaaaagccgcgtactacggtgggctggaaagggctgattaacgatccgcatatggataacagcttccagatcaacgacggtctgcgtattgcccgcaaattgctgctcgatattaacgacagcggtctgccagcggcgggtgaattcctggatatgatcaccctacaatatctcgctgacctgatgagctggggcgcaattggcgcacgtaccaccgaatcgcaggtgcaccgcgaactggcgtctggtctttcttgtccggtaggtttcaaaaatggcactgatggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcgcaggtgatgatcgatttcagccatgctaactcgtcaaaacaattcaaaaagcagatggatgtttgtactgacgtttgccagcagattgccggtggcgaaaaggccattattggcgtgatggtggaaagccatctggtggaaggcaatcagagcctcgagagcggggaaccgctggcctacggtaagagcatcaccgatgcctgcattggctgggatgataccgatgctctgttacgtcaactggcgagtgcagtaaaagcgcgtcgcggg

SEQ ID NO: 7 - DNA, 1059 bp, Artificial Sequence
  Description of Artificial Sequence Synthetic polynucleotide
atgctgttattcgagactgtgcgtgaaatgggtcatgagcaagtccttttctgtcatagcaagaatcccgagatcaaggcaattatcgcaatccacgataccaccttaggaccggctatgggcgcaactcgtatcttaccttatattaatgaggaggctgccctgaaagatgcattacgtctgtcccgcggaatgacttacaaagcagcctgcgccaatattcccgccgggggcggcaaagccgtcatcatcgctaaccccgaaaacaagaccgatgacctgttacgcgcatacggccgtttcgtggacagcttgaacggccgtttcatcaccgggcaggacgttaacattacgcccgacgacgttcgcactatttcgcaggagactaagtacgtggtaggcgtctcagaaaagtcgggagggccggcacctatcacctctctgggagtatttttaggcatcaaagccgctgtagagtcgcgttggcagtctaaacgcctggatggcatgaaagtggcggtgcaaggacttgggaacgtaggaaaaaatctttgtcgccatctgcatgaacacgatgtacaactttttgtgtctgatgtcgatccaatcaaggccgaggaagtaaaacgcttattcggggcgactgttgtcgaaccgactgaaatctattctttagatgttgatatttttgcaccgtgtgcacttgggggtattttgaatagccataccatcccgttcttacaagcctcaatcatcgcaggagcagcgaataaccagctggagaacgagcaacttcattcgcagatgcttgcgaaaaagggtattctttactcaccagactacgttatcaatgcaggaggacttatcaatgtttataacgaaatgatcggatatgacgaggaaaaagcattcaaacaagttcataacatctacgatacgttattagcgattttcgaaattgcaaaagaacaaggtgtaaccaccaacgacgcggcccgtcgtttagcagaggatcgtatcaacaactccaaacgctcaaagagtaaagcgattgcggcg

SEQ ID NO: 8 - DNA, 1656 bp, Artificial Sequence
  Description of Artificial Sequence Synthetic polynucleotide
atgcgcactccatattgtgtcgctgattacctgttggaccgcttgactgattgcggtgcagatcacctgttcggtgttccaggcgattacaacctgcaattcctggatcacgttatcgactcacctgacatttgctgggtcgggtgtgcgaatgagttaaacgcttcctatgcagcagacggctatgcacgctgtaaggggtttgcagcattgctgaccacttttggtgtgggagaattgagtgcaatgaacgggatcgcaggatcctatgctgagcatgtgccagtgttgcatatcgttggtgcaccaggtactgctgcccaacagcgtggtgagctgttgcatcacaccttaggtgatggtgagtttcgccacttctaccatatgagcgaacccattacagtcgctcaagctgttctgacggagcagaatgcgtgctacgaaatcgatcgtgttcttaccaccatgttgcgtgaacgtcgtcctgggtatctgatgttgccagcagatgtcgcgaagaaagcagccacaccaccagtcaatgcactgacacataagcaagctcacgcagattctgcctgtttgaaggcgtttcgtgatgcagctgagaacaagcttgccatgtcgaagcgtacggcacttcttgcggatttcctggttctgcgtcatggcctgaagcacgcacttcagaaatgggttaaggaagtgcctatggcacatgccacgatgcttatgggtaagggtatctttgacgaacgccaagcaggattctatggaacatacagtggaagcgcatccactggtgctgtcaaggaagccatcgaaggagcggatactgtgctttgcgttgggacccgtttcacagacacgttaacagctggatttacccaccaattaacgccagctcagaccattgaagtacaacctcatgcagcacgtgttggtgatgtctggttcactggtattcccatgaaccaagctatcgagacactggtcgaattgtgcaaacaacatgtccatgcaggcttgatgagctcgtcaagtggtgctattcccttcccacaaccagacggctccttgacgcaggagaacttctggcgtacccttcaaactttcattcgtccaggtgacatcattcttgcagatcaaggtacttcggcgtttggtgctattgacttgcgtcttccagctgacgtgaacttcattgtccagcctctgtggggttcgatcggatacactcttgctgcagcattcggtgcacaaacagcttgtcctaatcgtcgcgtaatcgtcctgactggtgacggtgcagctcagttgacaattcaggagttaggcagcatgttacgtgataagcaacacccaatcatcttggtcctgaacaacgagggttacactgtagagcgtgccatccatggtgcagaacaacgttacaacgacattgcgttgtggaattggacccatattccacaagctttaagcctggatccgcaaagtgagtgctggcgtgtatccgaagctgaacaactggcagatgtgctggagaaggtcgcacatcatgaacgcttatcgttgatcgaagtcatgttgcctaaggcagatattccaccattgttaggtgctctgacgaaagccttggaggcgtgtaacaacgcc

SEQ ID NO: 9 - DNA, 1491 bp, Artificial Sequence
  Description of Artificial Sequence Synthetic polynucleotide
atgcccactttgaaccttgatctgccgaacggcattaaatcgacgattcaggctgatttgttcatcaataacaagttcgtcccagcacttgatggcaagacatttgcgacaatcaatccatcgacaggaaaagaaatcggccaggtggcagaggcttccgcaaaggacgtggacctggcagttaaggctgcacgcgaagccttcgagaccacttggggtgagaacacaccaggtgatgcacgtggtcgcttgttgatcaaactggctgagcttgtggaggccaacattgacgagcttgcagcgattgaatccctggataatggtaaggctttctcaattgcaaagtccttcgatgtagctgcggttgctgccaacttacgttactatggtggctgggctgataagaaccatggtaaagtaatggaggtcgatacaaagcgtctgaactatacgcgtcacgagcccatcggagtctgtggacagatcattccatggaactttccactgctcatgtttgcttggaagttgggaccagccttggctactggcaatacgatcgtcctgaagactgcagagcaaacacctctgtcggctatcaagatgtgtgaattgatcgtggaggcaggatttcctccaggcgtagtaaatgtgatctcgggctttggtcctgtcgcaggagcagcgatctcacagcacatggacatcgataagatcgctttcacaggaagtaccctggtaggacgtaacatcatgaaggcagcagcatccacgaacctgaagaaggtcacgctggaattgggtggaaagagtcctaacatcatcttcaaggacgcggatttagatcaagctgtgcgttggagtgcgttcggaatcatgttcaatcacggccagtgctgctgtgcagggagtcgcgtgtatgtagaggagtccatctatgatgctttcatggagaagatgacggcacattgtaaggcactgcaagttggtgatcctttctccgcaaatacgttccagggtccgcaagtttcacaattgcaatatgaccgtatcatggagtacatcgaatctggcaagaaggacgcaaatctggcattgggaggcgtccgtaaggggaacgaaggttacttcatcgaaccgacaatctttactgacgtccctcatgatgcaaagattgctaaggaagagatctttggacctgtagtcgttgtatccaagtttaaggatgagaaagatttgatccgcatcgccaatgatagcatttatggccttgcagcagcagtcttctctcgcgacatttcacgtgcgatcgaaacagctcataagctgaaggcaggtacagtatgggttaactgctacaatcagttgatcccacaagttccattcggtgggtacaaagcgtccgggatcggtcgcgaattaggagaatatgcgttatctaactataccaatatcaaggcggtccatgttaatctgagtcaacctgctcccatt

SEQ ID NO: 10 - DNA, 25 bp, Artificial Sequence
  Description of Artificial Sequence Synthetic oligonucleotide
acctattggtataaggaggtaaata

SEQ ID NO: 11 - DNA, 32 bp, Artificial Sequence
  Description of Artificial Sequence Synthetic oligonucleotide
taggaggtttctatcaaattaaggaggttatt

SEQ ID NO: 12 - DNA, 42 bp, Artificial Sequence
  Description of Artificial Sequence Synthetic oligonucleotide
ggtaggacgattgtccggtagatttaaggaggattttttttt

SEQ ID NO: 13 - DNA, 50 bp, Artificial Sequence
  Description of Artificial Sequence Synthetic oligonucleotide
tcattttagtcaatatcagtattgattccatacacgtaaggaggttattt

SEQ ID NO: 14 - protein, 520 aa, Artificial Sequence
  Description of Artificial Sequence Synthetic polypeptide
MQTQKPTLELLTCEGAYRDNPTALFHQLCGDRPATLLLEFADIDSKDDLKSLLLVDSALRITALSDTVTIQALSGNGEALLTLLDNALPAGVENEQSPNCRVLRFPPVSPLLDEDARLCSLSVFDAFRLLQNLLNVPKEEREAMFFGGLFSYDLVAGFENLPQLSAENSCPDFCFYLAETLMVIDHQKKSTRIQASLFAPNEEEKQRLTARLNELRQQLTEAAPPLPVVSVPHMRCECNQSDEEFGGVVRLLQKAIRAGEIFQVVPSRRFSLPCPSPLAAYYVLKKSNPSPYMFFMQDNDFTLFGASPESSLKYDATSRQIEIYPIAGTRPRGRRADGSLDRDLDSRIELEMRTDHKELSEHLMLVDLARNDLARICTPGSRYVADLTKVDRYSYVMHLVSRVVGELRHDLDALHAYRACMNMGTLSGAPKVRAMQLIAEAEGRRRGSYGGAVGYFTAHGDLDTCIVIRSALVENGIATVQAGAGVVLDSVPQSEADETRNKARAVLRAIATAHHAQETF

SEQ ID NO: 15 - protein, 397 aa, Artificial Sequence
  Description of Artificial Sequence Synthetic polypeptide
MTTLLNPYFGEFGGMYVPQILMPALRQLEEAFVSAQKDPEFQAQFNDLLKNYAGRPTALTKCQNITAGTNTTLYLKREDLLHGGAHKTNQVLGQALLAKRMGKTEIIAETGAGQHGVASALASALLGLKCRIYMGAKDVERQSPNVFRMRLMGAEVIPVHSGSATLKDACNEALRDWSGSYETAHYMLGTAAGPHPYPTIVREFQRMIGEETKAQILEREGRLPDAVIACVGGGSNAIGMFADFINETDVGLIGVEPGGHGIETGEHGAPLKHGRVGIYFGMKAPMMQTEDGQIEESYSISAGLDFPSVGPQHAYLNSTGRADYVSITDDEALEAFKTLCLHEGIIPALESSHALAHALKMMRENPEKEQLLVVNLSGRGDKDIFTVHDILKARGEI

SEQ ID NO: 16 - protein, 452 aa, Artificial Sequence
  Description of Artificial Sequence Synthetic polypeptide
MQTVLAKIVADKAIWVETRKEQQPLASFQNEVQPSTRHFYDALQGARTAFILECKKASPSKGVIRDDFDPARIAAIYKHYASAISVLTDEKYFQGSFDFLPIVSQIAPQPILCKDFIIDPYQIYLARYYQADACLLMLSVLDDEQYRQLAAVAHSLEMGVLTEVSNEEELERAIALGAKVVGINNRDLRDLSIDLNRTRELAPKLGHNVTVISESGINTYAQVRELSHFANGFLIGSALMAHDDLNAAVRRVLLGENKVCGLTRGQDAKAAYDAGAIYGGLIFVATSPRCVNVEQAQEVMAAAPLQYVGVFRNHDIADVADKAKVLSLAAVQLHGNEDQLYIDNLREALPAHVAIWKALSVGETLPARDFQHIDKYVFDNGQGGSGQRFDWSLLNGQSLGNVLLAGGLGADNCVEAAQTGCAGLDFNSAVESQPGIKDARLLASVFQTLRAY

SEQ ID NO: 17 - protein, 531 aa, Artificial Sequence
  Description of Artificial Sequence Synthetic polypeptide
MADILLLDNIDSFTYNLADQLRSNGHNVVIYRNHIPAQTLIERLATMSNPVLMLSPGPGVPSEAGCMPELLTRLRGKLPIIGICLGHQAIVEAYGGYVGQAGEILHGKASSIEHDGQAMFAGLTNPLPVARYHSLVGSNIPAGLTINAHFNGMVMAVRHDADRVCGFQFHPESILTTQGARLLEQTLAWAQQKLEPTNTLQPILEKLYQAQTLSQQESHQLFSAVVRGELKPEQLAAALVSMKIRGEHPNEIAGAATALLENAAPFPRPDYLFADIVGTGGDGSNSINISTASAFVAAACGLKVAKHGNRSVSSKSGSSDLLAAFGINLDMNADKSRQALDELGVCFLFAPKYHTGFRHAMPVRQQLKTRTLFNVLGPLINPAHPPLALIGVYSPELVLPIAETLRVLGYQRAAVVHSGGMDEVSLHAPTIVAELHDGEIKSYQLTAEDFGLTPYHQEQLAGGTPEENRDILTRLLQGKGDAAHEAAVAANVAMLMRLHGHEDLQANAQTVLEVLRSGSAYDRVTALAARG

SEQ ID NO: 18 - protein, 268 aa, Artificial Sequence
  Description of Artificial Sequence Synthetic polypeptide
MERYESLFAQLKERKEGAFVPFVTLGDPGIEQSLKIIDTLIEAGADALELGIPFSDPLADGPTIQNATLRAFAAGVTPAQCFEMLALIRQKHPTIPIGLLMYANLVFNKGIDEFYAECEKVGVDSVLVADVPVEESAPFRQAALRHNVAPIFICPPNADDDLLRQIASYGRGYTYLLSRAGVTGAENRAALPLNHLVAKLKEYNAAPPLQGFGISAPDQVKAAIDAGAAGAISGSAIVKIIEQHINEPEKMLAALKAFVQPMKAATRS

SEQ ID NO: 19 - protein, 350 aa, Artificial Sequence
  Description of Artificial Sequence Synthetic polypeptide
MNYQNDDLRIKEIKELLPPVALLEKFPATENAANTVAHARKAIHKILKGNDDRLLVVIGPCSIHDPVAAKEYATRLLTLREELQDELEIVMRVYFEKPRTTVGWKGLINDPHMDNSFQINDGLRIARKLLLDINDSGLPAAGEFLDMITLQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCTDVCQQIAGGEKAIIGVMVESHLVEGNQSLESGEPLAYGKSITDACIGWDDTDALLRQLASAVKARRG

SEQ ID NO: 20 - protein, 353 aa, Artificial Sequence
  Description of Artificial Sequence Synthetic polypeptide
MLLFETVREMGHEQVLFCHSKNPEIKAIIAIHDTTLGPAMGATRILPYINEEAALKDALRLSRGMTYKAACANIPAGGGKAVIIANPENKTDDLLRAYGRFVDSLNGRFITGQDVNITPDDVRTISQETKYVVGVSEKSGGPAPITSLGVFLGIKAAVESRWQSKRLDGMKVAVQGLGNVGKNLCRHLHEHDVQLFVSDVDPIKAEEVKRLFGATVVEPTEIYSLDVDIFAPCALGGILNSHTIPFLQASIIAGAANNQLENEQLHSQMLAKKGILYSPDYVINAGGLINVYNEMIGYDEEKAFKQVHNIYDTLLAIFEIAKEQGVTTNDAARRLAEDRINNSKRSKSKAIAA

SEQ ID NO: 21 - protein, 552 aa, Artificial Sequence
  Description of Artificial Sequence Synthetic polypeptide
MRTPYCVADYLLDRLTDCGADHLFGVPGDYNLQFLDHVIDSPDICWVGCANELNASYAADGYARCKGFAALLTTFGVGELSAMNGIAGSYAEHVPVLHIVGAPGTAAQQRGELLHHTLGDGEFRHFYHMSEPITVAQAVLTEQNACYEIDRVLTTMLRERRPGYLMLPADVAKKAATPPVNALTHKQAHADSACLKAFRDAAENKLAMSKRTALLADFLVLRHGLKHALQKWVKEVPMAHATMLMGKGIFDERQAGFYGTYSGSASTGAVKEAIEGADTVLCVGTRFTDTLTAGFTHQLTPAQTIEVQPHAARVGDVWFTGIPMNQAIETLVELCKQHVHAGLMSSSSGAIPFPQPDGSLTQENFWRTLQTFIRPGDIILADQGTSAFGAIDLRLPADVNFIVQPLWGSIGYTLAAAFGAQTACPNRRVIVLTGDGAAQLTIQELGSMLRDKQHPIILVLNNEGYTVERAIHGAEQRYNDIALWNWTHIPQALSLDPQSECWRVSEAEQLADVLEKVAHHERLSLIEVMLPKADIPPLLGALTKALEACNNA

SEQ ID NO: 22 - protein, 497 aa, Artificial Sequence
  Description of Artificial Sequence Synthetic polypeptide
MPTLNLDLPNGIKSTIQADLFINNKFVPALDGKTFATINPSTGKEIGQVAEASAKDVDLAVKAAREAFETTWGENTPGDARGRLLIKLAELVEANIDELAAIESLDNGKAFSIAKSFDVAAVAANLRYYGGWADKNHGKVMEVDTKRLNYTRHEPIGVCGQIIPWNFPLLMFAWKLGPALATGNTIVLKTAEQTPLSAIKMCELIVEAGFPPGVVNVISGFGPVAGAAISQHMDIDKIAFTGSTLVGRNIMKAAASTNLKKVTLELGGKSPNIIFKDADLDQAVRWSAFGIMFNHGQCCCAGSRVYVEESIYDAFMEKMTAHCKALQVGDPFSANTFQGPQVSQLQYDRIMEYIESGKKDANLALGGVRKGNEGYFIEPTIFTDVPHDAKIAKEEIFGPVVVVSKFKDEKDLIRIANDSIYGLAAAVFSRDISRAIETAHKLKAGTVWVNCYNQLIPQVPFGGYKASGIGRELGEYALSNYTNIKAVHVNLSQPAPI

SEQ ID NO: 23 - DNA, 169 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgc